(19) United States
(12) Patent Application Publication
(10) Pub. No.: US 2025/0369050 A1
(43) Pub. Date: Dec. 4, 2025

(54) METHODS AND COMPOSITIONS RELATED TO ASSESSMENT AND TREATMENT OF KIDNEY DISEASE

(71) Applicant: Board of Regents, The University of Texas System, Austin, TX (US)

(72) Inventor: Kumar Sharma, San Antonio, PA (US)

(21) Appl. No.: 18/834,621

(22) PCT Filed: Feb. 4, 2023
(86) PCT No.: PCT/US2023/062009

§ 371 Date: Jul. 31, 2024

Related U.S. Application Data
(60) Provisional application No. 63/306,943, filed on Feb. 4, 2022.

(51) Int. Cl. C12Q 1/6883; C12N 15/113

(52) U.S. Cl. CPC C12Q 1/6883; C12N 15/1138; C12N 2310/14; C12Q 2600/106; C12Q 2600/118

(57) ABSTRACT

Embodiments described herein relate to methods and compositions for diagnosis, monitoring, classifying, staging and determination of treatment regimens in subjects with or at risk of kidney disease and/or all-cause mortality by determining the level of an amino acid or nucleic acid in a biological fluid, such as urine. In certain aspects the subjects are diagnosed with diabetes (or other underlying risk factor for kidney disease such as hypertension) and have normal levels of urine albumin. Additionally, disclosed herein are methods of treating a condition of fibrosis by inhibiting production or function of adenine.

### PRIORITY PARAGRAPH

This International Application claims priority to U.S. Provisional Application Ser. No. 63/306,943 filed Feb. 4, 2022 which is incorporated herein by reference in its entirety.

### STATEMENT REGARDING FEDERALLY FUNDED RESEARCH

This invention was made with government support under grant Nos. DK094352, DK110541, and DK114920 awarded by the National Institutes of Health. The government has certain rights in the invention.

### FIELD OF THE INVENTION

Embodiments of the invention are directed generally to the field of Medicine and Nephrology, in particular assessment of kidney function.

### BACKGROUND

The diagnosis of patients at risk of progressive reduction of kidney function with time is currently based on an increase in urine albumin levels above the normal range of 0-30 mg albumin/gram creatinine in a random or timed urine collection and a measure of their glomerular filtration rate (GFR). Albumin is an abundant protein produced primarily by the liver and present in the blood circulation. The blood albumin is prevented from entering the final urine via barriers at the glomerular level and by reabsorption of albumin at the tubular level. In many patients who have progressive kidney failure, there is a disruption of glomerular or tubular function and albumin “leaks” into the final urine. This is an important biomarker for progressive kidney disease and approximately 70% of patients who go on to need kidney replacement therapy have increased levels of albumin (standardized by urine creatinine to account for variations in concentration of the urine) in their urine prior to the need for kidney replacement therapy. The other main criteria to determine if someone has reduced kidney function is a reduction in the glomerular filtration rate, which is estimated based on the blood creatinine value. The normal estimated GFR is usually above 90 ml/min/1.73 m². The National Kidney Foundation (URL kidney.org/professionals/explore-your-knowledge/how-to-classify-ckd) defines stage G1A1 kidney disease as those with eGFR>90 ml/min/1.73 m² and normal or“high normal” levels of albumin in their urine or some other abnormality in their urine (e.g., blood cells in urine). Stage G1A2 patients have eGFR>90 ml/min/1.73 m² and moderately increased levels of albumin in their urine (30-300 mg/gram). These patients aretermed as having microalbuminuria. Stage G1A3 patients have eGFR>90 ml/min/1.73 m² and have severely increased levels of albumin in their urine (>300 mg/gram) and are also termed as having macroalbuminuria. Patients who need kidney replacement therapy typically have eGFR values below 10 ml/min/1.73 m².

GFR categories in CKD

| | GFR | |
|---|---|---|
| Category | ml/min/1.73 m² | Terms |
| G1 | ≥90 | Normal or high |
| G2 | 60-89 | Mildly decreased* |
| G3a | 45-59 | Mildly to moderately decreased |
| G3b | 30-44 | Moderately to severely decreased |
| G4 | 15-29 | Severely decreased |
| G5 | <15 | Kidney failure |

Abbreviations: CKD, chronic kidney disease; GFR, glomerular filtration rate.
*Relative to young adult level.
In the absence of evidence of kidney damage, neither GFR category G1 nor G2 fulfill the criteria for CKD.

Albuminuria categories in CKD

| CategoryACR (mg/g) | Terms |
|---|---|
| A1 <30 | Normal to mildly increased |
| A2 30-300 | Moderately increased* |
| A3 >300 | Severely increased** |

Abbreviations: ACR, albumin-to-creatinine ratio; CKD, chronic kidney disease.
*Relative to young adult level.
**Including nephrotic syndrome (albumin excretion ACR >2220 mg/g)

Over 120,000 patients will develop end-stage kidney disease requiring dialysis or transplantation each year. Typically, the patients who have progressive decline in their eGFR and will need kidney replacement therapy will have increased levels of albuminuria. In epidemiological studies, 70% of patients who develop end-stage renal disease (ESRD) will have high levels of albumin in their urine during the early stages of CKD (Stage 1-3). However, it has been estimated that approximately 30% of patients who go on to develop Stage 5 CKD do not have evidence of increased albuminuria during their earlier stages of CKD (Stage 1-3). In those patients with levels of urine albumin/creatinine ratio below 30 mg/gram the conclusion is that they are at low risk of developing kidney disease and therefore are usually not treated with medications to reduce progression of kidney disease, such as renin-angiotensin-aldosterone inhibitors or sodium-glucose transporter 2-inhibitors. The subset of patients with diabetes (or some underlying risk factor such as hypertension) and normoalbuminuria and have progressive decline in kidney function are difficult to identify. In addition, these normoalbuminuric diabetic kidney disease (NADKD) patients are often not included in clinical trials to determine if new therapies are beneficial for them. As the 30% of patients who develop end-stage kidney disease but do not have albuminuria, this is a large segment of the entire end stage kidney disease (ESKD) population that are currently not identified for interventional treatment as it is very difficult to identify which patients will go on to develop ESKD based on traditional clinical risk markers.

There is therefore a large unmet need for developing a non-invasive biomarker assay to diagnose and stratify patients for their risk of progressive kidney disease.

### SUMMARY

In some aspects, the invention described herein relates to methods and compositions for diagnosis, monitoring, classifying, staging and determination of treatment regimens in subjects with or at risk of kidney disease by determining the level of a nucleic acid, an amino acid, or both, in a biological fluid, such as blood or urine. In some instances, the nucleic acid measured can be a pyrimidine (e.g., cytosine, thymine), a purine (e.g., adenine, guanine, or uric acid), or uracil. In some instances, the amino acid is asparagine, aspartic acid, betaine, homocysteine, isoleucine, L-alpha-aminobutyric acid, lysine, methionine, nicotinic acid, ornithine, phenylalanine, pipecolate, threonine, tryptophan, tyrosine or valine. In some instances, the nucleic acid, nucleoside, or amino acid measured is a polyamine (e.g., adenine, ornithine, spermine, putrescine). In certain aspects the subjects are diagnosed with diabetes (or other underlying risk factor for kidney disease such as hypertension) and have normal levels of urine albumin. The level of adenine in a biological fluid, such as urine, is obtained from a patient and compared to the level of urine adenine with a control reference range or threshold value. An increased level of urine adenine (e.g., represented by urine adenine/creatinine ratio) in the sample indicates that the patient is at high risk of reduction of kidney function or at risk of kidney failure, independent of albuminuria levels. The urine adenine assay can independently diagnose normoalbuminuric diabetic kidney disease (NADKD) or microalbuminuric diabetic kidney disease and identify the patients at high risk for decline in kidney function or patients who will need future kidney replacement therapy. The urine adenine/creatinine ratio can also identify patients at high risk of all-cause mortality and kidney failure independent of albuminuria levels. The urine adenine assay and or a biological fluid adenine measurement (e.g., in blood samples) may also identify patients with other causes of kidney disease (such as hypertension, ischemic-related acute kidney injury) that are at risk of decline in kidney function and will have a need for kidney replacement therapy.

In some aspects, the present invention is directed to methods for prognosing or identifying patients, e.g., patients with diabetes or other risk factor for kidney disease, having normal or elevated levels of urine albumin. The methods can determine which patients are at risk of progressive kidney disease and the potential future need for kidney replacement therapy. Based on untargeted metabolomic analysis from spatial omics and urine metabolomics, the metabolite adenine was found to associate with glomerulosclerosis, tubular atrophy, tubulointerstitial fibrosis and vascular arteriosclerosis as well as decline in kidney function.

In some aspects, the present invention is directed to methods for prognosing or identifying patients, e.g., patients with diabetes or other risk factor for kidney disease, having normal or elevated levels of urine albumin. The methods can determine which patients are at risk of progressive kidney disease, the potential future need for kidney replacement therapy and increased mortality. Based on untargeted and targeted metabolomic analysis from spatial omics and urine metabolomics, the metabolite adenine was found to associate with glomerulosclerosis, tubular atrophy, tubulointerstitial fibrosis and vascular arteriosclerosis as well as decline in kidney function.

In some aspects, the present invention is directed to methods for the diagnosis of patients at risk of chronic kidney disease who have normal or have elevated levels of albumin in their urine and to methods for predicting the need for kidney replacement therapy based on the presence in a bodily fluid, such as urine or blood, of a level of adenine that exceeds a threshold level. The present invention is also directed to diagnostic assays to measure adenine levels in biofluids and useful to identify animal models of disease and identify which drugs may be beneficial in certain conditions of kidney disease.

In some aspects, the present invention is directed to methods of treating a condition of progressive fibrosis or cellular senescence in a subject, comprising administering a therapeutically effective amount of pharmaceutical composition to the subject, wherein the pharmaceutical composition inhibits production or function of adenine. In some instances, the pharmaceutical composition inhibits production of adenine by blocking a cellular signaling pathway leading to endogenous adenine production. In some instances, the pharmaceutical composition inhibits production of endogenous adenine by inhibiting expression or function of 5′-Methylthioadenosinephosphorylase (MTAP). In some cases, inhibiting expression of MTAP comprises using a short hairpin RNA targeting at least a portion of a polynucleotide sequence encoding MTAP. In some cases, inhibiting expression of MTAP comprises using a nuclease (e.g., Cas9 endonuclease) coupled to a guide RNA targeting at least a portion of a polynucleotide sequence encoding MTAP. In some cases, inhibiting expression of MTAP comprises inserting a silencer sequence near a polynucleotide sequence encoding MTAP. In some cases, inhibiting function of MTAP comprises using a MTAP neutralizing antibody. In some cases, inhibiting function of MTAP comprises using a small molecule inhibitor of MTAP. Small molecule inhibitors of MTAP can be MT-DADMe-ImmA (FIG. 17), or other small molecule inhibitors as described in U.S. Pat. No. 8,916,571 and U.S. Ser. No. 10/918,641 (both incorporated herein by reference). In some aspects, MTAP may be inhibited by sodium hydrogen sulfide or analogues of hydrogen sulfide or other molecules, including siRNA to MTAP, to affect levels or activity of MTAP. In some aspects, the cell surface receptor for adenine may be inhibited by G-Protein Coupled Receptor (GPCR) antagonists or siRNA. In some aspects, the cell surface insulin receptor may be responding to adenine and be inhibited by blockers of the insulin receptor. In some aspects, an siRNA targeting a portion of the insulin receptor inhibits the insulin receptor (IR). In some aspects, the pharmaceutical composition inhibits function of endogenous adenine by inhibiting expression or function of mTORC1. In some instances, the pharmaceutical composition is a mTORC1 inhibitor selected from the group consisting of rapamycin, a rapalog, a rapamycin derivative, temsirolimus, everolimus, umirolimus, zotarolimus, torin-1, torin-2, and vistusertib. In some cases, the pharmaceutical composition comprises an siRNA that inhibits function of endogenous adenine by inhibiting expression or function of PI3K. In some instances, the pharmaceutical composition is a small molecule PI3K inhibitor. In some cases, the small molecule PI3K inhibitor is LY294002. In some instances, the pharmaceutical composition inhibits function of endogenous adenine by inhibiting expression or function of AKT. In some instances, the pharmaceutical composition is a small molecule AKT inhibitor. In some cases, the small molecule AKT inhibitor is MK2202. In some cases, the pharmaceutical composition inhibits function of endogenous adenine by inhibiting expression or function of insulin receptor (IR). In certain embodiments, IR specific inhibitors include nucleic acids, proteins and small molecules. In certain embodiments, the IR specific inhibitor is a nucleic acid. In certain embodiments, the nucleic acid is an antisense compound. In certain embodiments, the modified oligonucleotide can be single stranded or double stranded. In certain embodiments, the nucleobase sequence of the modified oligonucleotide comprises a nucleobase sequence that is at least 70%, 75%, 80%, 85%, 90%, 95% or 100% identical to the nucleobase sequences recited in any one of SEQ ID NOs: 1-22. In certain embodiments, the nucleobase sequence of the modified oligonucleotide is at least 70%, 75%, 80%, 85%, 90%, 95% or 100% complementary to the nucleobase sequences recited in any one of SEQ ID NOs: 23-31. In some cases, the insulin receptor inhibitor is a knockdown mechanism comprising at least one of RNA interference (RNAi), small interfering RNA (siRNA), short hairpin RNA (shRNA), or a bacterial RNA-guided endonuclease directed towards the insulin receptor. Examples of siRNA targeting IR can comprise a nucleobase sequence in Table 10A and Table 11A (SEQ IDs. 1-22); however, it is contemplated that other IR siRNAs, including those commercially available can also be used. General definitions and concepts relating to siRNA are described in U.S. Pat. Nos. 11,377,658 and 10,364,433, both incorporated herein by reference. In some cases, the pharmaceutical composition inhibits function of endogenous adenine by inhibiting expression or function of Gi-coupled adenine receptor (AdeR).

In some aspects, the pharmaceutical composition inhibits function of endogenous adenine by activating AMP-activated protein kinase (AMPK). In some instances, the pharmaceutical composition is an AMPK activator selected from the group of NaHS, Metformin, AICAR, Metformin hydrochloride, A769662, RSVA405, ZLN024 hydrochloride, PT1, and PF06409577. In some instances, incorporation of the small molecule or siRNA or modified siRNA or biologic may be incorporated into a dendrimer or chitosan or other chemical entity to enhance distribution to target organs. In some instances, the condition of progressive fibrosis is at least one of kidney disease, liver disease, lung disease, cardiac fibrosis, brain fibrosis, neurodegenerative disease, joint fibrosis, skin fibrosis, myelofibrosis, retroperitoneal fibrosis. In some cases, the kidney disease is at least one of chronic kidney disease, diabetic kidney disease, end-stage renal disease (kidney failure), glomerulosclerosis, tubulointerstitial fibrosis, kidney arterial sclerosis, kidney arteriolar sclerosis, kidney neoplasia, and kidney tubular atrophy. In some cases, the lung disease is at least one of interstitial lung diseases (ILDs) and pulmonary fibrosis. It is further contemplated that treating the diseases described herein can be achieved by a combination of two or more methods disclosed herein.

Some aspects of the disclosure provide a method of identifying a subject at risk of a kidney disease comprising measuring a level of a nucleic acid, nucleoside, or amino acid in a biological sample (e.g., urine or blood) from the subject; and assessing the risk of the kidney disease based on the nucleic acid, nucleoside, or amino acid level as compared to a standard or reference. In some instances, the standard is the average value measured in a population of healthy individuals having normal kidney function. In some instances, the standard is previous measurements of the same subject. In some instances, the nucleic acid, nucleoside, or amino acid measured is adenine, cytosine, guanine, thymine, uracil, asparagine, aspartic acid, betaine, homocysteine, isoleucine, L-alpha-aminobutyric acid, lysine, methionine, nicotinic acid, ornithine, phenylalanine, pipecolate, threonine, tryptophan, valine, or any combination thereof. In some instances, the kidney disease is at least one of chronic kidney disease, diabetic kidney disease, end-stage renal disease (kidney failure), glomerulosclerosis, tubulointerstitial fibrosis, kidney arterial sclerosis, kidney arteriolar sclerosis, kidney neoplasia, and kidney tubular atrophy. In some instances, the kidney disease can include progressive decline in kidney function. In some cases, the risk of kidney disease comprises risk of disease progression. In some instances, the risk of kidney disease comprises risk of developing the kidney disease in the future. In some cases, the method disclosed herein further comprises processing a biological sample from the subject to separate or enrich the nucleic acid, nucleoside, or amino acid. In some instances, processing the biological sample comprises performing capillary electrophoresis, liquid chromatography (HPLC), capillary electrophoresis, liquid chromatograph, or any combination thereof. In some cases, measuring nucleic acid, nucleoside, or amino acid is performed by diode array detection (e.g., wavelengths between 200-400 nm), ZipChip™, mass spectrometry, electromagnetic radiation absorption, or any combination thereof. In some instances, an adenine level of greater than a threshold value is indicative of 1) decline in glomerular filtration rate (GFR), 2) CKD progression, and/or 3) kidney failure. In some cases, an adenine level lower than a threshold value of 2.92 nmol adenine/mmol creatinine indicates a low risk of developing a kidney condition or disease described herein. In some cases, an adenine level higher than a threshold value of 2.92 nmol adenine/mmol creatinine indicates an intermediate risk of developing a kidney condition or disease described herein. In some cases, an adenine level higher than a threshold value of 4.08 nmol adenine/mmol creatinine indicates an above-average risk of developing a kidney condition or disease described herein. In some cases, an adenine level higher than a threshold value of 5.23 nmol adenine/mmol creatinine indicates a high risk of developing a kidney condition or disease described herein. In some instances, an adenine level between 80 and 500 mg/g is indicative of ESKD. In some instances, the subject is diabetic. In some instances, the subject has normal albumin levels. In some instances, the subject is a mammal, for example, a human, a non-human primate, a rodent, a canine or feline. In some instances, the method disclosed herein further comprise administering a treatment to the subject, wherein the treatment comprises surgery, chemotherapy, radiation therapy, dietary restrictions, treatment of high blood pressure, treatment of diabetes, weight management, smoking cessation, treatment of high cholesterol and/or other lipid levels, kidney transplant, dialysis, administration of erythropoietin and/or calcitriol, diuretics, vitamin D, or phosphate binder or any combination thereof.

Some aspects of the disclosure provide a method of treating or preventing a kidney disease in a subject having or at risk of developing a kidney disease comprising (a) determining the level of adenine in a biological sample (e.g., urine or blood) from the subject; and (b) administering a treatment for the kidney disease if the adenine level is above a threshold (e.g., >2.92 nmol adenine/mmol creatinine or above the lowest tertile for a defined population). The tertiles found for low risk patients was 0-2.92 nmol adenine/mmol creatinine, for intermediate risk 2.92-5.23 nmol adenine/mmol creatinine and high risk was greater than 5.23 nmol adenine/mmol creatinine. In some instances, the subject is diabetic. In some instances, the subject has normal urine albumin levels. In some instances, the subject is human or an animal model for CKD.

Some aspects of the disclosure provide an assay for determining the level of adenine in a biological sample (e.g., urine or blood) from a subject comprising: (a) separating analytes in a sample forming sample fractions; and (b) quantifying adenine in the appropriate fractions. In some instances, the subject is diabetic. In some instances, the subject has normal albumin levels. In some instances, the subject is human or an animal model for CKD.

Some aspects of the disclosure provide a method of identifying a subject at risk for progressive reduction of kidney function comprising: (a) processing a biological sample (e.g., urine or blood) from the subject to separate adenine forming a processed sample; (b) measuring adenine levels in the processed sample; and (c) assessing the risk for progressive reduction of kidney function based on the adenine level as compared to a standard. In some cases, processing is selected from capillary electrophoresis, liquid chromatography (HPLC), or capillary electrophoresis and liquid chromatograph. In some cases, measuring adenine levels is by mass spectrometry, electromagnetic radiation absorption, or by ZipChip™. In some cases, adenine levels of greater than >2.92 nmol adenine/mmol creatinine is indicative of a subject at risk for progressive reduction of kidney function and/or all-cause mortality. In some cases, adenine levels of greater than 4.08 nmol adenine/mmol creatinine is indicative of a subject at an above-average risk for progressive reduction of kidney function and/or all-cause mortality. In some cases, adenine levels of greater than 5.23 nmol adenine/mmol creatinine is indicative of a subject at high risk for progressive reduction of kidney function and/or all-cause mortality. In some instances, the subject is diabetic. In some instances, the subject has normal urine albumin levels, low levels of urine albumin (microalbuminuria) or high levels of urine albumin (macroalbuminuria). In some instances, the subject is human or an animal model.

Some aspects of the disclosure provide a method for assaying a therapy for the treatment of kidney disease comprising: (a) exposing or contacting a subject with a test agent that is a potentially a therapy for kidney disease; and (b) monitoring the subject by quantitating adenine levels; wherein a reduction in adenine levels is indicative of a therapeutic benefit of the test agent for the treatment of kidney disease.

Some aspects of the disclosure provide a method for monitoring progression of CKD in a subject comprising: (a) obtaining a first biological sample from a subject at a first time point and a second biological sample at a second time point; (b) measuring adenine level in the first biological sample and the second biological sample; and (c) assessing CKD by comparing the difference in adenine levels between the first time point and the second time point.

Some aspects of the disclosure provide a method of identifying a subject at risk of a kidney disease comprising: (a) measuring adenine and creatinine levels in a biological sample from the subject; and (b) assessing the risk of the kidney disease based on the adenine/creatinine ratio as compared to a standard.

Some aspects of the disclosure provide a method of measuring a biological sample from a subject, comprising: (a) measuring a nucleic acid, nucleoside, or amino acid in the biological sample from the subject; (b) calculating the ratio of the nucleic acid, nucleoside, or amino acid to creatinine; and c) comparing the ratio to a standard to determine whether the ratio is greater than a threshold value. In some cases, the threshold value is about 2.92 nmol nucleic acid, nucleoside or amino acid/mmol creatinine. In some cases, the threshold value is about 4.08 nmol nucleic acid, nucleoside or amino acid/mmol creatinine. In some cases, threshold value is about 5.23 nmol nucleic acid, nucleoside or amino acid/mmol creatinine. In some cases, the nucleic acid, nucleoside, or amino acid is a purine, polyamine, adenine, cytosine, guanine, thymine, uracil, asparagine, aspartic acid, betaine, homocysteine, isoleucine, L-alpha-aminobutyric acid, lysine, methionine, nicotinic acid, omithine, phenylalanine, pipecolate, threonine, tryptophan, or valine, or any combination thereof. In some cases, an adenine level of greater than 2.92 nmol nucleic acid, nucleoside or amino acid/mmol creatinine is indicative of a subject at risk for developing a kidney condition or disease. In some cases, an adenine level of greater than 4.08 nmol nucleic acid, nucleoside or amino acid/mmol creatinine is indicative of a subject at an above-average risk for developing a kidney condition or disease. In some cases, an adenine level of greater than 5.23 nmol nucleic acid, nucleoside or amino acid/mmol creatinine is indicative of a subject at high risk for a kidney condition or disease. In some instances, the subject is diabetic. In some instances, the subject has normal urine albumin levels, low levels of urine albumin (microalbuminuria) or high levels of urine albumin (macroalbuminuria). In some instances, the subject is human or an animal model. In some cases, the kidney condition or disease is at least one of chronic kidney disease, diabetic kidney disease, hypertension-related kidney disease, glomerulonephritis-associated kidney disease, end-stage renal disease (kidney failure), glomerulosclerosis, tubulointerstitial fibrosis, kidney arterial sclerosis, kidney arteriolar sclerosis, kidney neoplasia, or kidney tubular atrophy.

Some aspects of the disclosure provide a method of measuring a biological sample from a subject, comprising: (a) measuring adenine and creatinine levels in the biological sample from the subject; (b) calculating the ratio of adenine/creatinine; and c) comparing the ratio of adenine/creatinine to a standard to determine whether the ratio is greater than about 2.92 nmol adenine/mmol, or about 4.08 nmol adenine/mmol creatinine, or about 5.23 nmol adenine/mmol creatinine. In some cases, an adenine level of greater than 2.92 nmol adenine/mmol creatinine is indicative of a subject at risk for developing a kidney condition or disease. In some cases, an adenine level of greater than 4.08 nmol adenine/mmol creatinine is indicative of a subject at an above-average risk for developing a kidney condition or disease. In some cases, an adenine level of greater than 5.23 nmol adenine/mmol creatinine is indicative of a subject at high risk for a kidney condition or disease. In some instances, the subject is diabetic. In some instances, the subject has normal urine albumin levels, low levels of urine albumin (microalbuminuria) or high levels of urine albumin (macroalbuminuria). In some instances, the subject is human or an animal model. In some cases, the kidney condition or disease is at least one of chronic kidney disease, diabetic kidney disease, hypertension-related kidney disease, glomerulonephritis-associated kidney disease, end-stage renal disease (kidney failure), glomerulosclerosis, tubulointerstitial fibrosis, kidney arterial sclerosis, kidney arteriolar sclerosis, kidney neoplasia, or kidney tubular atrophy.

Certain embodiments are directed to methods for detecting a level of urine adenine in the upper two tertiles (e.g., between 2.92 and 5.23 nmol adenine/mmol creatinine for intermediate risk, and above 5.23 nmol adenine/mmol creatinine for high risk) of developing end-stage kidney disease.

Other embodiments are directed to methods for detecting a level of urine adenine/creatinine ratio in the upper two tertiles, which indicates a high risk of mortality and end-stage kidney disease.

Other embodiments are directed to methods for detecting a level of urine adenine in the upper two tertiles (e.g., between 2.92 and 5.23 nmol adenine/mmol creatinine for intermediate risk, and above 5.23 nmol adenine/mmol creatinine for high risk) of progressive decline in kidney function as measured by GFR.

Still other embodiments are directed to methods for detecting a level of urine adenine indicative of which patients should be selected for certain classes of medications to reduce kidney disease progression, e.g., >2.92 nmol adenine/mmol creatinine, >4.08 nmol adenine/mmol creatinine, or 5.23 nmol adenine/mmol creatinine.

Certain embodiments are directed to assay methods to be used in patients with diabetes with no albuminuria as part of a screen to identify those at risk of kidney disease.

In other embodiments, the assays are used to monitor patients and identify if they are responding to new treatment regimens for kidney protection.

In still further embodiments, the assay can identify animal models of kidney disease relevant to the human condition.

The term “biomarker” as used herein, refers to any biological compound related to the progressive development of chronic kidney disease. For example, a biomarker may comprise adenine, or any of its metabolites or derivatives. In certain aspects the biomarker is adenine.

Adenine is one of the two purine nucleobases (the other being guanine) used in forming nucleotides. Adenine has the following chemical structure:

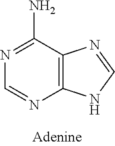

Creatinine is a breakdown product of creatine phosphate from muscle and protein metabolism. It is released at a constant rate by the body (depending on muscle mass). Creatinine has the following chemical structure:

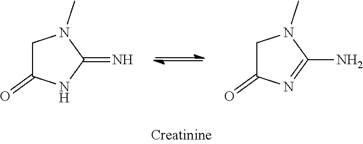

Some aspects of the disclosure provide a method of treating a condition of progressive fibrosis in a subject, comprising administering a therapeutically effective amount of a pharmaceutical composition to the subject, wherein the pharmaceutical composition is at least one of an inhibitor of adenine accumulation, an inhibition of adenine receptor, an inhibitor of adenine signaling, or any combination thereof. In some embodiments, the inhibitor of adenine accumulation is at least one of MTAP inhibitor (MTDIA) or hydrogen sulfide. In some embodiments, the inhibitor of adenine receptor is at least one of Gi-coupled adenine receptor (AdeR) or insulin receptor. In some embodiments, the inhibitor of adenine signaling is at least one of Akt inhibitor, PI3K inhibitor, or mTOR inhibitor.

A “biological sample” as used herein is a sample of biological fluid. Examples of biological samples are blood, blood fractions, plasma, serum, or urine. Furthermore, also pools or mixture of the above-mentioned samples may be employed. A biological sample may be provided by collecting a sample from a subject but can also be provided by using a previously collected sample. In a preferred embodiment, a urine or blood sample is taken from the subject. In one embodiment, a first sample is obtained from the subject prior to initiation of a therapeutic treatment.

A biological sample from a patient means a sample from a subject suspected to be affected by a disease. As used herein, the term “subject” refers to any mammal, including both human and other mammals. Preferably, the methods of the present invention are applied to human subjects.

The terms “treating” or “treatment” refer to any success or indicia of success in the attenuation or amelioration of an injury, pathology or condition, including any objective or subjective parameter such as abatement, remission, diminishing of symptoms or making the injury, pathology, or condition more tolerable to the patient, slowing in the rate of degeneration or decline, making the final point of degeneration less debilitating, improving a subject's physical or mental well-being, or prolonging the length of survival. The treatment or amelioration of symptoms can be based on objective or subjective parameters, including the results of a physical examination, neurological examination, and/or psychiatric evaluations.

“Effective amount” and “therapeutically effective amount” are used interchangeably herein and refer to an amount of an antibody or functional fragment thereof, as described herein, effective to achieve a particular biological or therapeutic result such as, but not limited to, the biological or therapeutic results disclosed herein. A therapeutically effective amount of the antibody or antigen-binding fragment thereof may vary according to factors such as the disease state, age, sex, and weight of the individual, and the ability of the antibody or functional fragment thereof to elicit a desired response in the individual. Such results may include, but are not limited to, the treatment of cancer, as determined by any means suitable in the art.

The term “prognosis” as used herein, refers to a medical conclusion based upon an analysis any biomarker that provides information regarding the progression of medical conditions including, but not limited to, chronic kidney disease. Such information includes but is not limited to the determination of risk for developing end-stage kidney disease or determine risk of progressive decline in kidney function.

The phrase “kidney disease” as used herein indicates any disease or condition that affects the kidneys such as, for example, chronic kidney disease, acute kidney disease, congenital kidney disease, polycystic kidney disease, hypertensive kidney disease, inflammatory kidney disease, glomerulonephritis, tubulo-interstitial disease, and the like. Chronic kidney disease often manifests in such a way that there are no detectable symptoms until there is irreversible damage to the kidneys.

The term “chronic kidney disease (CKD)” as used herein, refers to a medical condition wherein exemplary symptoms may include, but are not limited to, hyperphosphatemia (i.e., for example, >4.6 mg/dl) or low glomerular filtration rates (i.e., for example, <90 ml/minute per 1.73 m² of body surface). However, many CKD patients may have normal serum phosphate levels in conjunction with a sustained reduction in glomerular filtration rate for 3 or more months, or a normal GFR in conjunction with sustained evidence of a structural abnormality of the kidney. Alternatively, “chronic kidney disease” refers to a medical condition wherein a patient has either (i) a sustained reduction in GFR <60 ml/min per 1.73 m² of body surface for 3 or more months; or (ii) a structural or functional abnormality of renal function for 3 or more months even in the absence of a reduced GFR. Structural or anatomical abnormalities of the kidney could be defined as but not limited to persistent microalbuminuria or proteinuria or hematuria or presence of renal cysts.

The terms “patient”, “individual” or “subject” are used interchangeably herein, and is meant a mammalian subject to be treated, for example, a human. In some cases, the processes of the present technology find use in experimental animals, in veterinary application, and in the development of vertebrate models for disease, including, but not limited to, rodents including mice, rats, and hamsters; birds, fish reptiles, and primates.

The terms “normal subject” and “healthy subject” refer to a mammalian subject, for example, a human, that is not or has not suffered from kidney disease and does not have a history of past kidney disease.

The term “glomerular filtration rate (GFR)” as used herein, refers to a measurement capable of determining kidney function. In general, normal glomerular filtration rates range between approximately 90-120 ml/minute per 1.73 m² of body surface. Compromised kidney function is assumed when glomerular filtration rates are less than 90 ml/minute per 1.73 m² of body surface. Kidney failure is probable when glomerular filtration rates fall below approximately 30 ml/minute per 1.73 m² of body surface.

The “estimated glomerular filtration rate, (eGFR)” is a measure of how well your kidneys are working. Your eGFR is an estimated number based on a blood test and your age, sex, body type and race.

A marker level can be compared to a reference level representing the same marker. In certain aspects, the reference level may be a reference level from control or non-diseased subject(s). Alternatively, reference level may be a reference level from a different subject or group of subjects. The reference level may be a single value or may be a range of values. In some embodiments, the reference level is an average level determined from a cohort of subjects.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, and platforms described herein are modular and not limited to sequential steps. Accordingly, terms such as “first” and “second” do not necessarily imply priority, order of importance, or order of acts.

As used herein, the term “about” when referring to a number or a numerical range means that the number or numerical range referred to is an approximation within experimental variability (or within statistical experimental error), and the number or numerical range may vary from, for example, from 1% to 10% of the stated number or numerical range. Unless otherwise indicated by context, the term “about” refers to t10% of a stated number or value.

As used herein, the term “approximately” means within an acceptable error range for the particular value as determined by one of ordinary skill in the art, which will depend in part on how the value is measured or determined, e.g., the limitations of the measurement system. For example, “approximately” can mean within 1 or more than 1 standard deviation, per the practice in the given value. Where particular values are described in the application and claims, unless otherwise stated the term “approximately” should be assumed to mean an acceptable error range for the particular value.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. All language such as “up to,” “at least,” “greater than,” “less than,” and the like include the number recited and refer to ranges which can be subsequently broken down into sub-ranges as discussed above. Finally, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Whenever the term “at least,” “greater than,” “greater than or equal to”, or a similar phrase precedes the first numerical value in a series of two or more numerical values, the term “at least,” “greater than,” “greater than or equal to” or similar phrase applies to each of the numerical values in that series of numerical values. For example, “at least 1, 2, or 3” is equivalent to “at least 1, at least 2, and/or at least 3.”

As used herein, the symbol “>” means “greater than”; the symbol “<” means “less than”; the symbol “>=” means “greater than or equal to”; the symbol “<=” means “less than or equal to.”

Whenever the term “no more than,” “less than,” “less than or equal to,” “no greater than,” “at most” or a similar phrase, precedes the first numerical value in a series of two or more numerical values, the term “no more than,” “less than,” “less than or equal to,” “no greater than,” “at most,” or similar phrase applies to each of the numerical values in that series of numerical values. For example, “less than 3, 2, or 1” is equivalent to “less than 3, less than 2, and/or less than 1.”

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1B. (A) Enrichment analysis of gene, protein, and metabolite markers for proximal tubule predicts well know functions. (B) Integration the tubulointerstitial metabolites into the KWEGG pathway Purine Metabolism (as identified in A). Enzyme commission numbers were mapped to the human gene products, multiple gene products of the same family were collapsed on family names. Selected reactions are shown. ACP3: acid phosphatase 3. ADP/AMP adenosine diphosphate/monophosphate. AK3: nucleoside-triphosphate-adenylate kinase. APRT: adenine phosphoribosyltransferase. D-R 1-P: D-Ribose 1-phosphate. NDP/NTP: Nucleoside 2/3-phosphate. NT: 5′-nucleotidase. Pi: Orthophosphate. PP: Diphosphate, reaction are reversible. PNP: purine-nucleoside phosphorylase. PRPP: 5-phospho-alpha-D-ribose 1-diphosphate.

FIG. 2A-2C. Endogenous adenine localized to regions of early glomerulosclerosis, atrophic tubules and vascular arteriosclerosis in human kidney biopsy (A). MALDI-MSI was performed on a patient with CKD and the metabolite adenine was found to co-localize with glomeruli that had lesions of early glomerulosclerosis (B) and adjacent to regions of atrophic tubules. Adenine was also localized to peritubular regions and overlaid the vascular wall of arteriosclerotic blood vessels (C).

FIG. 3. ZipChip™ Urine adenine assay correlates well LC-MS/MS. A ZipChip™ urine adenine assay was developed and found to highly correlate with urine adenine measured by LC-MS/MS. N=23 samples, r=0.90, p<0.0001.

FIG. 4. Flowchart of biopsy study in Pima Native American population (adapted from PMID 30830355).

FIG. 5. Flow Diagram for Analysis Steps and Brief Overview of Results.

FIG. 6 Forest plot (hazard ratios with 95% CIs) of ESKD risk of 15 untargeted and targeted metabolites.

FIG. 7 Adenine dose-dependently reduced ATP generation in human proximal tubular cells. At high doses, adenine exposure caused complete reduction of ATP generation in HK2 cells by Seahorse analysis. ****p<0.0001.

FIG. 8. Urine adenine correlates with glomerulosclerosis in PIMA Indian population.

FIG. 9. Urine adenine identifies patients with ESKD.

FIG. 10. Forest Plot showing subgroup analysis indicate that Males and those with high urine adenine/creatinine ratio (urine AdCR) have a higher rate of developing ESKD across different albuminuria categories. HR Males (1.11 95% CI 1.05-1.17, p<0.001, Female 0.99, 95% CI 0.92-1.06, p=0.8). Macroalbuminuria HR 1.04, 0.98-1.10, p=0.2), Microalbuminuria 1.13, CI 1.02-1.25, p=0.022, and Normoalbuminuria HR 1.15, CI 1.00-1.33, p=0.048).

FIG. 11A. Image of ZipChip equipment.

FIG. 11B. Microfluidic chip in ZipChip cartridge.

FIG. 12. Plasma adenine identifies animal model could be used for modeling acute kidney injury. Plasma adenine was measured by Zipchip-QExactive method in mice 24 h post sham surgery or 24 h post ischemia-reperfusion injury. There is a significant increase in plasma adenine in the IRI mice indicating they would be a good model for mimicking human kidney disease.

FIG. 13. Adenine is toxic to kidney cells (Causative of disease).

FIG. 14. Role of MTAP in Adenine Production.

FIG. 15. Endogenous production of Adenine and Effect of Adenine is central to kidney disease progression.

FIG. 16. MTAP is increased in IR-35 (acute) and MDM2 KO (chronic) kidney disease.

FIG. 17. Structure of MTAP inhibitor used in experiment.

FIG. 18. Protective effect of MTAP inhibitor. A small molecule inhibitor of MTAP MT-DADMe-ImmA (Synonyms: MTDIA; Methylthio-DADMe-Immucillin) protects kidney cells from cell death and will therefore be protective to progressive kidney disease.

FIG. 19. Effect of Rapamycin and NaHS on Adenine. Adenine-induced fibronectin accumulation was ameliorated by inhibition of mTORC1 and activation of AMPK. A. Kidney tubules (MCT) cells were pre-incubated with 10 nM of rapamycin for 30 min followed by incubation with 20 μM of adenine for 15 min. B. MCT cells were incubated with 20 μM of adenine and 250 μM NaHS for 15 min. Inhibition of mTOR (Mammalian target of rapamycin) via Rapamycin blocks adenine-induced fibronectin production in kidney cells which leads to scarring of kidney cells (panel A). The compound sodium hydrogen sulfide (NaHS) can also inhibit adenine-induced fibronectin production (panel B) by inhibiting mTOR and/or activating AMP-activated protein kinase (AMPK).

FIG. 20. High urine adenine/creatinine ratio (tertile 3 vs tertile 1) at baseline associates with ESKD in non-macroalbuminuric diabetic patients. High urine adenine/creatinine levels (tertile 3 vs tertile 1) identify patients with increased risk of ESKD in a US cohort including Caucasians, African Americans and Hispanics (n=551) with diabetes and non-macroalbuminuria.

FIG. 21. Mortality and ESKD risk is highest in diabetic patients with top tertiles of urine adenine/creatinine. A US cohort including Caucasians, African Americans and Hispanics (n=904) demonstrates that top two urine adenine tertiles identify patients at higher risk of ESKD and mortality as compared to lowest tertile.

FIG. 22. Urine adenine/creatinine ratio is higher in type 1 diabetic (T1D) patients with hyperfiltration vs T1D patients with normfiltration. Difference in baseline euglycemic adenine between hyperfiltrating and normofiltrating participants. Significant p-value stated. *p<0.05.

FIG. 23. Empagliflozin reduces urine adenine levels in patients with hyperfiltration. Hyperfilterers (red lines and dots) and normofilterers (blue lines and dots). Overall effect of treatment (Visit 3 and 4 vs. Visit 12 and 13) on least square mean changes to log-transformed adenine levels is reported. Significant p-values stated. *p<0.05, **p<0.01.

FIG. 24. Adenine rapidly stimulates Type I collagen in kidney tubular cells.

FIG. 25. MTAP inhibitor DADMe-ImmA given once before ischemia-reperfusion injury prevents an increase in BUN levels in mice undergoing ischemia-reperfusion injury. Mice underwent ischemia for 30m followed by reperfusion and 24 h later, the BUN was measured. In mice treated with the MTAP inhibitor there was no significant increase in BUN whereas mice treated with vehicle had a marked increase in BUN.

FIG. 26. MTAP levels, fibronectin and urine ACR are increased in the db/db kidney disease and reduced by NaHS. NaHS administered for 4 weeks to db/db mice and db/m mice and MTAP/-b-actin and fibronectin/b-actin levels were evaluated by immunoblotting. Urine ACR was measured at end of 4-week period of NaHS administration. There was a significant reduction in MTAP (A), fibronectin (B) and urine ACR (C) with NaHS administration (n=8-10/group, **p<0.01, ***p<0.001).

FIG. 27. A specific siRNA for the insulin receptor (IR) completely blocks adenine induced stimulation of mTOR and fibronectin in kidney tubular cells. An siRNA for IR completely blocks IR levels in kidney tubular cells (A) and prevented phosphorylation of S6K (B). The siRNA for IR blocks adenine-induced fibronectin production in kidney tubular cells (C).

FIG. 28. Inhibition of PI3K with LY294002, or AKT with MK2202 or mTOR with Rapamycin all block Adenine-induced stimulation of mTOR pathway. S6K phosphorylation (P-S6K) is a sensitive indicator of mTOR stimulation and was increased within minutes of adenine challenge in tubular cells.

FIG. 29. Spatial levels of adenine are increased in human diabetic kidneys. Quantitative assessment across healthy controls (n=5) and diabetic samples (n=8 T1D and n=8 T2D) with MALDI-MSI demonstrated a statistical increase of adenine in kidney tissue sections.

FIG. 30. Adenine administration to mice increases serum sTNFR1 (A), plasma KIM-1 (B). (n=12 in control group and n=7 in adenine treated group, *p<0.05, **p<0.01, ***p<0.001, ****p<0.001 vs control by t-test or One-way ANOVA).

FIG. 31. Adenine administration to mice increases kidney (A) and heart (B) hypertrophy and mTOR mediated matrix molecules in the kidney (C). Quantitative assessment of phosphor-S6 kinase as a measure of mTOR (D), fibronectin/-b-actin (E) and collagen type 1 alpha 2 chain (F). (n=12 in control group and n=7 in adenine treated group, *p<0.05, **p<0.01, ***p<0.001, ****p<0.001 vs control by t-test or One-way ANOVA).

FIG. 32. MTAP inhibitor DADMe-ImmA for 8 weeks reduces urine ACR, urine H2O2 and urine collagen in db/db mice. Control (db/m) and diabetic (db/db) mice were treated from week 12 to week 20 with MTAP inhibitor (10 mg/kg/day in drinking water) and 24 h urine samples were collected and measured for albumin/creatinine ratio (A), hydrogen peroxide/creatinine ratio (B) and collagen/creatinine ratio (C). (*p<0.05, ANOVA, n=5-6/group)

FIG. 33. MTAP inhibitor DADMe-ImmA has a beneficial effect to reduce kidney hypertrophy and inhibit kidney matrix accumulation in diabetic kidney. Control (db/m) and diabetic (db/db) mice were treated from week 12 to week 20 with MTAP inhibitor (10 mg/kg/day in drinking water) and kidney weight was standardized per tibia length (A). Kidney cortex was immunoblotted for fibronectin and laminin y1 and standardized for b-actin with quantitative data shown in (B) and (C), respectively. (*p<0.05, ANOVA, n=5-6/group)

FIG. 34. siRNAs to insulin receptor (IR) based on sequence in mouse and rat reduces IR in mouse kidney cells. Mouse cortical tubular (MCT) cells were reverse transfected with siRNAs targeting mouse and rat insulin receptor mRNA (m/r IRsiRNA #1 and m/r IR siRNA #5) using RNAiMax (6 ul in optiMEM) and plated on 6 well plates. After 72 h of incubation, total RNA was isolated and subjected to cDNA synthesis followed by qRTPCR (n=6/group, ****p<0.0001). There was a significant reduction of mouse insulin receptor at mRNA level with both IR siRNA #1 and IRsiRNA #5.

FIG. 35. In vivo administration of IRsiRNA #1 reduced IR specifically in mouse kidney and prevented adenine-induced rise in BUN levels in mice. Mice were administered vehicle or kidney targeting IRsiRNA #1 prior to being challenged with adenine (5 mM) in the drinking water for 4 days. At 4 days, mouse kidney showed reduction of IR in mouse kidney cortex, as compared to control sample, but not in liver as demonstrated by immunoblotting with antibody to IR and b-actin (representative immunoblot, upper panel). The mice given vehicle and adenine (n=2) had an elevation in BUN levels of 3-fold vs non-adenine group (lower panel). The mice treated with siRNA for IR had no increase in BUN levels despite exposure to adenine (n=2) (lower panel).

FIG. 36. siRNA to Human and Pig Insulin receptor is effective to reduce mRNA for Insulin receptor in human kidney cells. Human kidney (HK2) proximal tubular cells were transfected with siRNA (4 ul from 10 um stock) using RNAiMax (6 ul in optiMEM) and plated on 6 well plates. After 48 h, the media was replaced with fresh media. After 96 h of the transfection, total RNA was isolated and subjected to cDNA synthesis followed by qRTPCR. There was a significant reduction of human insulin receptor in HK2 cells after transfection with H/P siRNA #2 and H/P IR siRNA #4 as compared to control siRNA (n=6/group, ****p<0.0001).

### DESCRIPTION

Methods and compositions are described for diagnosis, monitoring, classifying, staging, and determination of treatment regimens in subjects by determining the level of adenine in a biological fluid, such as urine, obtained from a patient and comparing the level of urine adenine with a control reference range or threshold value. In certain aspects the subject has diabetes (or other underlying risk factor for kidney disease such as hypertension) and normal levels of urine albumin. An increased level of urine adenine in the sample identifies a patient as at high risk of reduction of kidney function or at risk of kidney failure or at risk of mortality. The urine adenine assay can independently diagnose normoalbuminuric diabetic kidney disease (NADKD) and identify the patients at high risk for decline in kidney function or patients who will need future kidney replacement therapy. The urine adenine assay and or a biological fluid adenine measurement (e.g., in blood samples) may also identify patients with other causes of kidney disease (such as hypertension, ischemic-related acute kidney injury) and are at risk of decline in kidney function and will have a need for kidney replacement therapy or at increased risk of mortality.

Herein, assay methods are described for adenine that can measure adenine in biological fluids. The urine assay identifies those patients with diabetes and normoalbuminuria and normal GFR (>90 ml/min/1.73 m²) who will have progression of kidney disease as measured by decline in GFR. Further, the urine assay identifies those diabetic patients with normoalbuminuria and reduced eGFR (25-75 ml/min/1.73 m² or Stage G2A1 and Stage G3A1 CKD) who are at increased risk for kidney failure (eGFR<15 ml/min/1.73 m², on dialysis or other renal replacement therapy).

### I. Kidney Injury and/or Disease

The kidneys are positioned in the back of the upper abdomen at either side of the spinal column. They are deep within the abdomen and are protected by the spine, lower rib cage, and the strong muscles of the back. This location protects the kidneys from many external forces. Kidneys are highly vascular organs, which means that they have a large blood supply. If injury occurs, severe bleeding may result.

Kidneys may be injured by damage to the blood vessels that supply or drain them. This may be in the form of aneurysm, arteriovenous fistula, arterial blockage, or renal vein thrombosis. The extent of bleeding depends on the location and the degree of injury. Kidneys may also bleed profusely if they are damaged centrally (on the inside)—this is a life-threatening injury. Fortunately, most kidney injuries caused by blunt trauma occur peripherally, only causing bruising of the kidney (usually a self-limiting process).

Each kidney filters about 1700 liters of blood per day and concentrates fluid and waste products into about 1 liter of urine per day. Because of this, the kidneys receive more exposure to toxic substances in the body than almost any other organ. Therefore, they are highly susceptible to injury from toxic substances. Analgesic nephropathy is one of the most common types of toxic damage to the kidney. Exposure to lead, cleaning products, solvents, fuels, or other nephrotoxic chemicals (those which can be toxic to the kidney) can damage kidneys. Excessive buildup of body waste products, such as uric acid (that can occur with gout or with treatment of bone marrow, lymph node, or other disorders) can also damage the kidneys.

Chronic Kidney Failure (CKF)—Unlike acute renal failure, chronic renal failure slowly gets worse. It most often results from any disease that causes gradual loss of kidney function. It can range from mild dysfunction to severe kidney failure. The disease may lead to end-stage renal disease (ESRD).

Chronic renal failure usually occurs over a number of years as the internal structures of the kidney are slowly damaged. In the early stages, there may be no symptoms. In fact, progression may be so slow that symptoms do not occur until kidney function is less than one-tenth of normal.

Chronic renal failure and ESRD affect more than 2 out of 1,000 people in the United States. Diabetes and high blood pressure are the two most common causes and account for most cases. Other major causes include, but are not limited to, Alport syndrome, Analgesic nephropathy, Glomerulonephritis of any type (one of the most common causes), Kidney stones and infection, Obstructive uropathy, Polycystic kidney disease, or Reflux nephropathy. Chronic renal failure results in an accumulation of fluid and waste products in the body, leading to a buildup of nitrogen waste products in the blood (azotemia) and general ill health. Most body systems are affected by chronic renal failure.

Initial symptoms may include, but are not limited to, fatigue, frequent hiccups, general ill feeling, generalized itching (pruritus), headache, nausea, vomiting, or unintentional weight loss. Further, later symptoms may include, but are not limited to, Blood in the vomit or in stools, decreased alertness, including drowsiness, confusion, delirium, or coma, decreased sensation in the hands, feet, or other areas, easy bruising or bleeding, increased or decreased urine output, Muscle twitching or cramps, seizures, or white crystals in and on the skin (uremic frost).

### II. Adenine—A Kidney Disease Progression and Mortality Biomarker

Certain patients present with normoalbuminuria and diabetes while having underlying kidney pathology. The diagnosis of kidney disease in a diabetic patient with normoalbuminuria and normal GFR is difficult. One approach is to demonstrate by kidney biopsy that there are pathologic features of kidney disease in a patient with normoalbuminuria and normal levels of GFR. This was accomplished in a prior trial of the Pima Native American population summarized below. Patients underwent a protocol with a research kidney biopsy after collection of urine samples.

Pima Indian study—All patients from this study were enrolled in a randomized clinical trial (Weil et al. 2013) testing the renoprotective efficacy of losartan vs. placebo in early diabetic kidney disease (ClinicalTrials.gov number, NCT00340678). Glomerular filtration rate (GFR) was measured annually throughout the trial by the urinary clearance of iothalamate. At the end of the six-year clinical trial, participants underwent percutaneous kidney biopsy to determine whether treatment was associated with preservation of kidney structure. Stored urine samples were available for 62 participants who collected urine prior to the kidney biopsy from the clinical trial who also had biopsy data available. The clinical trial was approved by the Institutional Review Board of the National Institute of Diabetes and Digestive and Kidney Diseases (NIDDK). Each participant signed an informed consent document. As described herein, a follow up biomarker study was conducted that was restricted to analysis of these participants that were randomized to placebo who were not taking any renin angiotensin system blocker at the time of biopsy or at the time of collection of either urine specimen used for measurement of urine adenine concentration.

CRIC Cohort. A metabolomics sub-study of the Chronic Renal Insufficiency Cohort (CRIC) was also used. The parent CRIC Study recruited (from 2003 on) a racially diverse group aged 21 to 74 years, ˜50% diabetic, with a broad range of kidney function [Feldman et al. J Am Soc Nephrol. 2003, 14(7 Suppl 2):S148-53]. Informed consent was obtained from participants; protocols were approved by IRBs and Scientific and Data Coordinating Center (approval #59 807882). The current study analyzed the urine metabolome at study entry (baseline) of 995 randomly selected CRIC participants with diabetes across CKD stages 3a, 3b, and 4, eGFR 45-60, 30-45, and 20-30 ml/min/1.73 m², respectively.

Adenine was identified as a biomarker for kidney disease progression. Various methods can be used to detect and/or measure adenine in a sample. In some embodiments the processing of a sample or sample mixture can involve separation. For example, a sample mixture comprising analytes to be detected can be prepared. In order to analyze the sample mixture, components of the sample mixture can be separated and analysis performed on only a fraction of the sample mixture. In this way, the complexity of the analysis can be substantially reduced since separated analytes can be individually analyzed thereby increasing the sensitivity of the analysis process. Of course, the analysis can be repeated one or more time on one or more additional fractions of the sample mixture to thereby allow for the analysis of all fractions of the sample mixture.

Adenine was identified as a biomarker for all-cause mortality and kidney failure in patients with diabetes and eGFR >20 ml/min/1.73 m2. Various methods can be used to detect and/or measure adenine in a sample. In some embodiments the processing of a sample or sample mixture can involve separation. For example, a sample mixture comprising analytes to be detected can be prepared. In order to analyze the sample mixture, components of the sample mixture can be separated and analysis performed on only a fraction of the sample mixture. In this way, the complexity of the analysis can be substantially reduced since separated analytes can be individually analyzed thereby increasing the sensitivity of the analysis process. Of course, the analysis can be repeated one or more time on one or more additional fractions of the sample mixture to thereby allow for the analysis of all fractions of the sample mixture.

The separation can be performed by chromatography. For example, liquid chromatography/mass spectrometry (LC/MS) can be used to effect such a sample separation and mass analysis. Moreover, any chromatographic separation process suitable to separate the analytes of interest can be used. For example, the chromatographic separation can be normal phase chromatography, reversed-phase chromatography, ion-exchange chromatography, size exclusion chromatography or affinity chromatography.

The separation can be performed electrophoretically. Non-limiting examples of electrophoretic separations techniques that can be used include, but are not limited to, 1D electrophoretic separation, 2D electrophoretic separation and/or capillary electrophoretic separation.

Liquid chromatography (LC) is a well-established analytical technique for separating components of a fluidic mixture for subsequent analysis and/or identification, in which a column, microfluidic chip-based channel, or tube is packed with a stationary phase material that typically is a finely divided solid or gel such as small particles with diameter of a few microns. The small particle size provides a large surface area that can be modified with various chemistries creating a stationary phase. A liquid eluent is pumped through the liquid chromatographic column (“LC column”) at a desired flow rate based on the column dimensions and particle size. This liquid eluent is sometimes referred to as the mobile phase. The sample to be analyzed is introduced (e.g., injected) in a small volume into the stream of the mobile phase prior to the LC column. The analytes in the sample are retarded by specific chemical and/or physical interactions with the stationary phase as they traverse the length of the column. The amount of retardation depends on the nature of the analyte, stationary phase and mobile phase composition. The time at which a specific analyte elutes or comes out of the end of the column is called the retention time or elution time and can be a reasonably identifying characteristic of a given analyte especially when combined with other analyzing characteristics such as the accurate mass of a given analyte. Or in other words, the analytes interact with the stationary phase based on the partition coefficients for each of the analytes. The partition coefficient is defined as the ratio of the time an analyte spends interacting with the stationary phase to the time spent interacting with the mobile phase. The longer an analyte interacts with the stationary phase, the higher the partition coefficient and the longer the analyte is retained on the LC column. An isocratic flow in LC describes a mobile phase of a constant composition. In contrast to this is the so called “gradient elution”, which is a separation where the mobile phase composition changes during a separation process. For example, a 20-minute gradient starts from 10% MeOH and ends up with 30% MeOH within 20 minutes.

Detection of analytes separated on an LC or nanoLC column can be accomplished by use of a variety of different detectors. Spectroscopic detectors rely on a change in refractive index, ultraviolet and/or visible light absorption, or fluorescence after excitation with a suitable wavelength to detect the separated components. Additionally, the separated components may be passed from the liquid chromatographic column into other types of analytical instruments for further analysis, e.g., liquid chromatography-mass spectrometry (LC/MS or LC-MS) separates compounds chromatographically before they are introduced to the ion source of a mass spectrometer.

The purpose of the LC column is to separate analytes such that a unique response (e.g., a UV absorption peak) for each analyte from a chosen detector can be acquired for a quantitative or qualitative measurement. The ability of a LC column to generate a separation is determined by the dimensions of the column and the particle size supporting the stationary phase. The retention time of an analyte can be adjusted by varying the mobile phase composition and the partition coefficient for an analyte. Increases in chromatographic separation can be achieved via a reduction in the LC column diameter, increasing LC column length and/or a reduction of stationary phase particle dimensions.

Mass spectrometry (“MS” or “mass-spec”) is an analytical technique used to measure the mass-to-charge ratio of gas phase ions. This is achieved by ionizing the sample and separating ions of differing masses and recording their relative abundance by measuring intensities of ion flux. A typical mass spectrometer comprises three parts: an ion source, a mass analyzer, and a detector system. The ion source is the part of the mass spectrometer that ionizes the substance under analysis (the analyte). The ions are then transported by magnetic or electric fields to the mass analyzer that separates the ions according to their mass-to-charge ratio (m/z). Many mass spectrometers use two or more mass analyzers for tandem mass spectrometry (MS/MS). The detector records the charge induced or current produced when an ion passes by or hits a surface. A mass spectrum is the result of measuring the signal produced in the detector when scanning m/z ions with a mass analyzer.

Mass spectrometry has rapidly developed as an important emerging method for the characterization of proteins. The two primary methods for ionization of whole proteins are electrospray ionization (ESI) and matrix-assisted laser desorption/ionization (MALDI). In keeping with the performance and mass range of available mass spectrometers, two approaches are used for characterizing proteins. In the first, intact proteins are ionized by either of the two techniques described above, and then introduced to a mass analyzer. In the second, proteins are enzymatically digested into smaller peptides using an agent such as trypsin or pepsin. The collection of peptide products are then introduced to the mass analyzer. The latter is often referred to as the “bottom-up” approach of protein analysis.

Analytes of interest to biological researchers are usually part of a very complex mixture of other molecules that co-exist in the biological medium. The high complexity of biological mixtures often makes coupling a separation technique, such as high performance liquid chromatography (HPLC), highly desirable or even required. In addition, HPLC on-line connected to ESI-MS offers the possibility for pre-concentration of dilute samples, desalting and removal of detergents. In many applications, and especially where relatively small volumes of sample are under analysis, improving detection sensitivity can become especially important. Improvement of detection sensitivity using concentration sensitive detectors such as UV/Vis absorbance and ESI mass spectrometers can be achieved by employing HPLC columns with smaller internal diameters (i.d.). For example, increased sensitivity during analysis can result from using nano-LC (e.g., column i.d. of 50-100 μm) and capillary LC (e.g., column i.d. of 320 μm). Flow rate of the mobile phase through such columns is from several nanoliters per minute (nL/min), to several microliters per minute (μL/min), and the mobile phase can be sprayed directly without post-column splitting. The process of electrospray ionization at flow rates on the order of nanoliters (“nL”) per minute has been referred to as “nanoelectrospray ionization” (nanoESI).

Electrospray ionization (ESI) or nanoESI is a commonly applied ionization technique when dealing with biomolecules such as peptides and proteins. The electrospray process creates highly-charged droplets that, under evaporation, create ions representative of the species contained in the solution. An ion-sampling orifice of a mass spectrometer may be used to sample these gas phase ions for mass analysis. When an electric potential or field is applied to the outlet of a conducting needle (often referred to as a sprayer or emitter) relative to an extracting electrode, such as one provided at the ion-sampling orifice of a mass spectrometer, the electric field generated on the needle causes the separation of positively and negatively charged ions in solution and pushes ions of one polarity (e.g., positively charged or negatively charged) to the solution surface. The higher the electric field is the greater the surface charge repulsion force that counteracts the fluid surface tension is. When the repulsion force of the solvated ions exceeds the surface tension of the fluid being electrosprayed, a volume of the fluid is pulled into the shape of a cone, known as a Taylor cone, which extends from the tip of the needle. A liquid jet extends from the tip of the Taylor cone and becomes unstable and generates charged-droplets. These small charged droplets are drawn toward the extracting electrode, e.g., the sampling electrode of a mass spectrometer. The small droplets are highly-charged and solvent evaporation from the droplets results in the excess charge in the droplet residing on the analyte molecules in the electrosprayed fluid. The charged molecules or ions are drawn through the ion-sampling orifice of the mass spectrometer for mass analysis. The potential voltage (“V”) required to initiate an electrospray is dependent on the size of the sprayer, the surface tension of the solution, and the electric field can be on the order of approximately 10⁶ V/m. The physical size of the needle and the fluid surface tension determines the density of electric field lines necessary to initiate electrospray.

In so-called nanoelectrospray, the sample is sprayed from a needle with a tip diameter less than about 5 μm, using a sample flow rate between 5 nL/min and 50 nL/min, for example. Charged droplets with diameters less than 1 micron can be formed at flow rates less than 40 nL/min. These small, highly-charged droplets can provide more efficient ionization of analytes contained within the droplets due to higher surface-to-volume ratios and smaller radii through which analytes need to diffuse to reach the charged surface of the droplets compared to conventional ESI. NanoESI-MS can thus be used for analyzing small amounts of sample with low sample concentrations (e.g., femtomole/microliter). Moreover, with nanoESI, the ion response for analytes contained in a sample solution is proportional to its concentration instead of its total amount. What this means is that if a solution is being sprayed at 200 nL/min or 50 nL/min or 20 nL/min the signal intensity as measured using mass spectrometry would be the same. Thus reducing a flow rate by a factor of 5 roughly increases mass spectrometry scans to be acquired for the same amount of sample by a factor of 5. As a result, signal averaging from the increased number of scans improves signal-to-noise ratios and ion statistics which enable multiple MS/MS experiments on the analytes and high accuracy in identifying analytes.

Tandem mass spectrometry (MS/MS) is a popular experimental method for identifying biomolecules such as proteins. Tandem MS involves multiple steps of mass selection or analysis, usually separated by some form of fragmentation. A tandem mass spectrometer is capable of multiple stages of mass spectrometry. For example, one mass analyzer can isolate one peptide from many others entering a mass spectrometer. A second mass analyzer then stabilizes the peptide ions while they collide with a gas, causing them to fragment by collision-induced dissociation (CID). A third mass analyzer then characterizes the fragments produced from the peptides. Tandem MS can also be done in a single mass analyzer over time as in a quadrupole ion trap. There are various methods for fragmenting molecules for tandem MS, including collision-induced dissociation (CID), electron capture dissociation (ECD), electron transfer dissociation (ETD), infrared multiphoton dissociation (IRMPD) and blackbody infrared radiative dissociation (BIRD).

Some specific non-limiting examples for detecting and/or measuring adenine in a sample include the following:

### A. ZipChip™-QExactive Assay for Urine Adenine

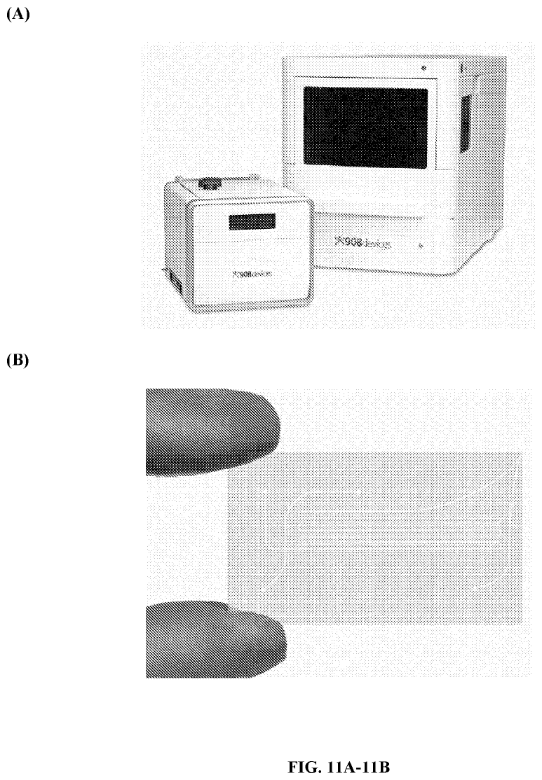

Adenine was assayed by using a quantitative capillary electrophoresis (CE) coupled to mass spectrometry method (ZipChip™-QExactive; Thermo Scientific Cat. No. 00950-01-00492 and 0726030). The ZipChip™ equipment (FIG. 11A) is a capillary electrophoresis separation method on a microfluidic device (FIG. 11B). This allows for rapid sample preparation, zone electrophoresis, is highly resistant to sample matrix effects, does not require analyte labeling, can use small sample volumes (2-20 μl) and allows for high throughput analysis (80-90 samples per day). The ZipChip™ platform was connected to a Thermo QC-Exactive for mass spectrometer detection of specific metabolites. An amino acid protocol was adapted to specifically measure urine adenine.

Briefly as a non-limiting example, for the ZipChip™ assay, 10 μL of urine sample or calibration standard mixture was mixed with 90 μL of extraction solution containing 80% methanol, 100 mM ammonium acetate and 1 μM of stable isotope labeled internal standards (including adenine) in a 96 well plate. After mixing, samples or calibration standards were kept in a −20° C. freezer for at least one hour and then centrifuged at 5000 rpm for 5 minutes. A 50 μL supernatant was transferred to a clean 96 well plate for analysis. Metabolite separation was achieved with a microfluidic chip which integrates capillary electrophoresis (CE) with nano-electrospray ionization through ZipChip™ interface (908 Devices, Boston, MA). For each sample, 20 μL solution was placed into the sample well and subsequently injected into the HS chip (Thermo Scientific Cat. No. 00950-01-00498) using a 10-s load time. A field strength of 1000V/cm was applied for separation over 4 minutes using a background electrolyte solution consisting of 2% formic acid in 50% methanol in water. The mass spectrometry acquisition was done with Q-Exactive™ mass spectrometer (Thermo, San Jose, CA). The resolving power was 17,500 with an AGC target of 3×10⁶, maximum injection time of 20 ms, and scan range of 75-500 m/z. Thermo Scientific's software Xcalibur-Quan Browser was used for quantitative data processing. Calibration curves for all metabolites were included with each plate. The targeted assay metabolite data were normalized to urine creatinine measured at the CRIC Central Laboratory or with creatinine measured by ZipChip™-QExactive™ method if creatinine not available.

ZipChip-QExeactive data: intra-assay and inter-assay variability. The urine adenine/creatinine ratio was measured in pooled human urine samples on two separate days with replicates of 4 on each day. The intra-assay CV was <5% on 4 replicate samples performed on the same day (% CV of 4.5% on day 1 or 4.2% on day 2). Inter-day QC CV was 7.4%.

### Urine Adenine Mg/Gram Creatinine

| | | | | |
|---|---|---|---|---|
| day 1 QC_1 | 2.107 | 2.142 | Avg. | Day1 |
| day 1 QC_2 | 2.072 | 0.098 | STDEV | |
| day 1 QC_3 | 2.104 | 4.554 | % CV | |
| day 1 QC_4 | 2.287 | | | |
| day 2 QC_5 | 2.331 | 2.405 | Avg. | Day2 |
| day 2 QC_6 | 2.342 | 0.101 | STDEV | |
| day 2 QC_7 | 2.551 | 4.210 | % CV | |
| day 2 QC_8 | 2.397 | | | |
| Avg. | 2.274 | | | |
| STDEV | 0.168 | | | |
| % CV | 7.387 | | | |

### B. LC-MS/MS Method for Urine Adenine

Sample Extraction: Calibration samples, urine samples were prepared in 96 well plate in mix solvent of 80:20 MeOH:H₂O. Internal standard was added at the concentration of 1 μM for the final volume of 200 μL. Multipipetting was performed in each well. Plate was covered with covering sheet and was placed in −20° C. for at least hr. The plate was then centrifuged at 4° C. with speed of 9 for 10 min. The plate was slowly removed. The cover detached and 100 μl of the top solution was taken and transferred to LCMS vial with insert for LCMS analysis. LC vials was centrifuged at room temperature at 9000 rpm for 20 min before analysis by LCMS using normal phase Chromatography with mobile phase water (A) and methanol (B) both contain 0.5% formic acid.

LC-MS/MS method and Calibration curve: The LC-MS/MS method was optimized for detection of adenine using rat urine standard samples. Multipoint calibration curve was performed for both adenine at the level of 1 nM to 40 μM. Linear instrument response with polynomial correlation coefficient of 0.99 was achieved for this range. Data was acquired in PRM mode as well as SIM modes simultaneously. Calibration curve of area of selected MS/MS fragments relative to internal standard versus standard concentration was used for adenine quantification in urine.

Assay correlation between Zipchip and LC/MSMS: As LC-MS/MS is considered a gold standard for measuring non-protein chemical entities in body fluids, the ZipChip™ method was compared to the LC-MS/MS method. Urine adenine from urine samples of control and diabetic rats (n=23 samples) were acquired by Zipchip™ and LC/MSMS to evaluate the correlation between the methods. Pearson Correlation and t test were determined using all concentration data of controls and ZDF samples. There was a very good correlation between the ZipChip™ and LC-MS/MS methods (r²=0.82 and p<0.00001).

### C. HPLC Assay for Urine Adenine

To avoid the use of a mass spectrometer an HPLC protocol can be developed to enable use of the most cost-effective instrumentation. The HPLC-protocol is tested vs the gold standard of LC-MS/MS and the ZipChip™ QExactive™ method described herein. The brief protocol is described as follows:

Adenine and creatinine will be identified and quantified using HPLC/UPLC equipped with DAD/UV detector. High-performance liquid chromatography (HPLC) is an analytical chemistry technique used to separate, identify, and quantify each component in a mixture. It utilizes pumps to pass a pressurized liquid solvent containing the sample mixture through a column filled with a solid adsorbent material. Each chemical species in the sample interacts differently with the adsorbent material in the column, causing different flow rates for the different components and leading to the separation of the components as they flow out of the column. This HPLC protocol to measure adenine and creatinine will provide advantages over a mass spectrometry-based protocol by requiring less expensive equipment and less highly-trained personnel to implement the protocol.

As a non-limiting example, small volume of solutions (5-20 μL) containing standards in the range of 1 μM to 100 μM will be used to establish the efficient HPLC/UPLC method. Metabolites are separated using a reverse phase column such as Hypersil™ C-18, 5-Am particle size, 250 mm*4.6 mm column (or any equivalent column), and a step gradient at the flow rate of 0.9 mL/min using two buffers and tetrabutylammonium hydroxide as the pairing reagent. The starting HPLC/UPLC condition may be considered as buffer A (12 mM tetrabutylammonium hydroxide, 10 mM KH₂PO₄, 0.125% methanol, pH 7.00), buffer B (2.8 mM tetrabutylammonium hydroxide, 100 mM KH₂PO₄, 30% methanol, pH 5.50) with a step gradient starts from 100% buffer A at 2 min and end up to 0% buffer A at 30 min. The separated analytes will be detected by a highly sensitive UV/DAD detector wavelengths from 200 and 400 nm. 7-(β-Hydroxyethyl)theophylline will be used as internal standard and spiked to the biological samples to correct for the loss of analyte during sample preparation. To establish an efficient method, the gradient and concentration of the buffer may need to be adjusted to achieve adequate separation for adenine and creatinine. Methods will be validated by the recovery test of the spiked standards in the filtered and deproteinated urine and plasma samples (200 μL) as well as calibration curve and the linearity response of the method in the range of 0, 10 nM to 500 μM for each metabolite. Internal standard (10 μM) will be used in the solution of standards and biological samples. The curve linearity will be evaluated by plotting the area under the spectra versus the concentration of each metabolite. Intra/Inter-assay precision will be performed by analyzing three replicates of quality control samples with two different concentrations in the same (for intra-assay) or separate days (for inter-assay) with same method. With adequate recovery and calibration linearity, the HPLC/UPLC method can be applied on biological samples. Biological samples will be diluted in DI water 10 times before being filtered by a membrane filter such as MCE (Mixed Cellulose Ester, 0.22 μm pore size) syringe filter or other efficient techniques. Aliquot of the filtered biological (100 μL) will be transferred in HPLC vials with insert. Samples will be stored in −80° C. if they are not analyzed immediately. Biological samples will be analyzed using HPLC/UPLC method in the following proposed sequence of couple of blanks, first series of calibration curve, biological samples, second series of calibration curve samples. Data will be acquired in terms of the area under the absorbance spectra and the wavelength at the maximum of the peak. The multipoint calibration curves for individual standards will be constructed based on the relative area of standard over internal standard versus the concentration of standards. Calibration curve equation will be used to estimate the unknown concentration of metabolites of interest in urine and plasma. Any dilution factor will be considered in quantitation.

### III. Treatments for Kidney Disease

Some treatment methods comprise (i) administering a drug to a subject in one or more administrations (e.g., 1, 2, 3, 4, 5, 6, 7, 8, 9 or 10 doses), (ii) determining the presence, absence or amount of a biomarker in or from the subject after (i), (iii) providing an indication of increasing, decreasing or maintaining a subsequent dose of the drug for administration to the subject, and (iv) optionally administering the subsequent dose to the subject, where the subsequent dose is increased, decreased or maintained relative to the earlier dose(s) in (i). In some embodiments, presence, absence or amount of a biomarker is determined before, after, or before and after each dose of drug has been administered to the subject, and sometimes presence, absence or amount of a biomarker is not determined after each dose of the drug has been administered (e.g., a biomarker is assessed after one or more of the first, second, third, fourth, fifth, sixth, seventh, eighth, ninth or tenth dose, but not assessed every time after each dose is administered).

An indication for adjusting a subsequent drug dose can be considered a need to increase or a need to decrease a subsequent drug dose. An indication for adjusting or maintaining a subsequent drug dose can be considered by a clinician, and the clinician may act on the indication in certain embodiments. In some embodiments, a clinician may opt not to act on an indication. Thus, a clinician can opt to adjust or not adjust a subsequent drug dose based on the indication provided.

An indication of adjusting or maintaining a subsequent drug dose, and/or the subsequent drug dosage, can be provided in any convenient manner. An indication may be provided in tabular form (e.g., in a physical or electronic medium) in some embodiments. For example, a biomarker threshold may be provided in a table, and a clinician may compare the presence, absence or amount of the biomarker determined for a subject to the threshold. The clinician then can identify from the table an indication for subsequent drug dose. In certain embodiments, an indication can be presented (e.g., displayed) by a computer after the presence, absence or amount of a biomarker is provided to computer (e.g., entered into memory on the computer). For example, presence, absence or amount of a biomarker determined for a subject can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount. A subsequent dose can be determined based on certain factors other than biomarker presence, absence or amount, such as weight of the subject, one or more metabolite levels for the subject (e.g., metabolite levels pertaining to liver function) and the like, for example.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

In one embodiment, the treatment comprises surgery, chemotherapy, radiation therapy, dietary restrictions, treatment of high blood pressure (for example, with angiotensin converting enzyme inhibitors (ACEIs) or angiotensin II receptor antagonists), treatment of diabetes (for example with sodium-glucose linked transporter 2-inhibitors (SGLT2-i), weight management, smoking cessation, treatment of high cholesterol and/or other lipid levels, kidney transplant, dialysis, administration of erythropoietin and/or calcitriol, diuretics, vitamin D, or phosphate binder or a combination thereof. In one embodiment, the subject is administered bardoxolone methyl, olmesartan medoxomil, sulodexide, and avosentan.

### IV. Kits

In another aspect, the present invention provides kits for quantifying adenine and qualifying kidney disease status, which kits are used to detect and quantify adenine. In one embodiment, the kit comprises a support, such as a chip, a microtiter plate or a bead or resin.

In a further embodiment, such a kit can comprise instructions for suitable operational parameters in the form of a label or separate insert.

### V. Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example I

High-Throughput Metabolomics and Diabetic Kidney Disease Progression: Evidence from the Chronic Renal Insufficiency (CRIC) Study

Metabolomics could offer novel prognostic biomarkers and elucidate mechanisms of diabetic kidney disease (DKD) progression. Using the diverse CRIC sample from 995 CRIC participants with diabetes, a high-throughput untargeted assay, followed by targeted analysis and rigorous statistical analysis to reduce false discovery, several novel metabolites implicated in DKD progression were identified. These findings could inform risk stratification and treatment strategies for patients with DKD. Urine samples (N=995) were assayed for relative metabolite abundance by untargeted flow-injection mass spectrometry (MS), and stringent statistical criteria were used to eliminate noisy compounds, resulting in 698 annotated metabolite ions. Utilizing the 698 metabolites' ion abundance along with clinical data (demographics, blood pressure, HbA1c, eGFR and albuminuria), we developed univariate and multivariate models for eGFR (estimated glomerular filtration rate) slope using penalized (lasso) and random forest models. Final models were tested on time-to-ESKD (End-stage-kidney-disease) via cross-validated C-statistics. Pathway enrichment analysis and a targeted analysis of a subset of metabolites were also conducted. Six eGFR slope models selected 9-30 variables. In the adjusted ESKD model with highest C-statistic, valine (or betaine) and 3-(4-Methyl-3-pentenyl) thiophene were associated (p<0.05) with 44% and 65% higher hazard of ESKD per doubling of metabolite abundance, respectively. Also, 13 (of 15) prognostic amino acids, including valine and betaine were confirmed in the targeted analysis. Enrichment analysis revealed pathways implicated in kidney and cardiometabolic disease.

### A. Research Design and Methods

Study Cohort. A metabolomics sub-study of the Chronic Renal Insufficiency Cohort (CRIC) was used. The parent CRIC Study recruited (from 2003 on) a racially diverse group aged 21 to 74 years, ˜50% diabetic, with a broad range of kidney function [Feldman et al. J Am Soc Nephrol. 2003, 14(7 Suppl 2):S148-53]. Informed consent was obtained from participants; protocols were approved by IRBs and Scientific and Data Coordinating Center (approval #59 807882). The current study analyzed the urine metabolome at study entry (baseline) of 995 randomly selected CRIC participants with diabetes across CKD stages 3a, 3b, and 4, eGFR 45-60, 30-45, and 20-30 ml/min/1.73 m², respectively.

Disease outcomes. Two outcomes were evaluated: annual rate of eGFR change (eGFR slope) and time-to-ESKD. EGFR-slope was estimated via mixed models using serial eGFR measures as described previously [Kwan et al. Am J Kidney Dis. 2020; 76(4):511-20]. Time-to-ESKD was the time from entry to CRIC Study to incident kidney failure with need for renal replacement therapy or kidney transplantation; drop-out or death before kidney failure were considered censoring events.

The CKD-EPI equation was used to calculate eGFR [Levey et al., Ann Intern Med 2009; 150(9):604-12]. Results were consistent (data not shown) when we repeated analyses using the CRIC-eGFR equations [Anderson et al. Am J Kidney Dis. 2012; 60(2):250-61], so findings are reported using the widely used CKD-EPI equation.

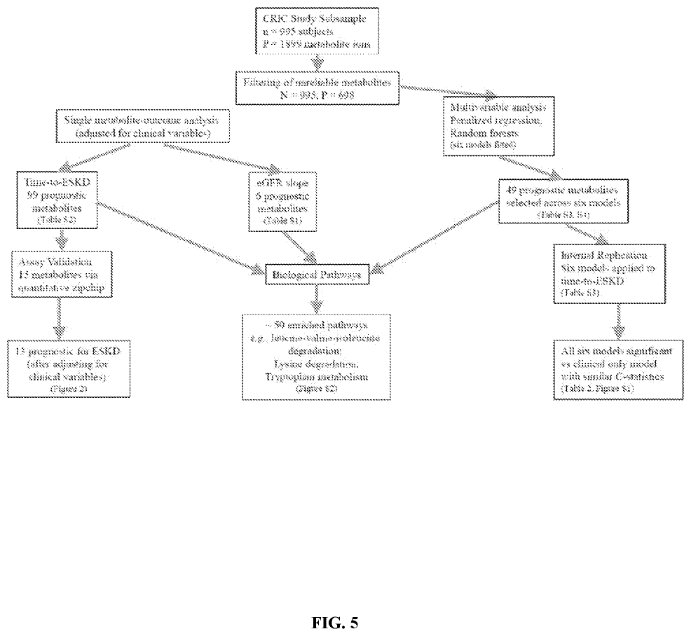

Statistical Analysis—Overview of analytic steps. (FIG. 5) Stringent filtering was implemented to exclude metabolite ions with high noise or low biological variability. Single metabolite associations were tested with DKD outcomes, correcting for multiple comparisons, with and without clinical variables adjustment. Next, multivariate metabolite models were developed, using statistical and machine learning methods to identify marker signatures associated with eGFR slope, and further tested these models on time-to-ESKD. A unique aspect of the approach is that we did not train models on the ESKD outcome; thus ESKD results serve as “internal replication” for a long-term clinical outcome. Finally, pathway enrichment analyses were conducted to investigate biological underpinnings of selected metabolites, and conducted a targeted analysis of a subset of metabolites.

Filtering metabolite ions. Leveraging technical replicates, QC and CRIC samples were used to eliminate ions with poor reliability in the untargeted analysis. Of 1899 annotated metabolite ions, the 698 which passed filtering criteria, are the final metabolite ion set for all subsequent analyses. A single ion could annotate multiple metabolites; these resulting ambiguities are clarified as appropriate.

Associations of single metabolite with outcomes. Associations between each of 698 log 2-transformed metabolite ions and outcomes were tested, with and without adjustment for clinical variables: age, gender, race, smoking, baseline BMI, mean arterial pressure (MAP), HbA1c, eGFR, albuminuria [Kwan et al. Am J Kidney Dis. 2020; 76(4):511-20]. For eGFR slope, Pearson correlations were calculated and fit linear models; for ESKD, Cox models were fit. Benjamini-Hochberg False Discovery Rate (FDR) was used to correct for multiple comparisons [Benjamini and Hochberg J Royal Statist Soc, Series B. 1995; 57:289-300].

Multivariate models for eGFR slope. Using eGFR slopes as outcome, penalized regression (via Lasso) and machine learning (via random forest) models, were developed to elicit multivariate prognostic metabolomic signatures. The Lasso reduces overfitting by imposing a penalty (λ) [Hastie et al. The elements of statistical learning: data mining, inference, and prediction. 2nd ed. NYC: Springer; 2009]. Two λ values chosen by 10-fold cross-validation were considered: λ·min, the value yielding the lowest prediction error; λ·Ise, the value within one SD of lowest prediction error. Four Lasso models were fit; each included 698 ions and 9 clinical variables as covariates. Two models forced all 9 clinical variables to be included, utilizing either λ·min or λ·Ise. The other two models did not force the clinical variables to be included. For random forests, percent increase of mean squared error were used to order variable importance [Hastie et al. The elements of statistical learning: data mining, inference, and prediction. 2nd ed. NYC: Springer; 2009]; for comparability, the same number of variables were selected as the corresponding Lasso models (without forcing clinical variables). Thus, six multiple metabolite ion models were fit: four lasso and two random forest models, each of which selected an optimal predictor set. As a final sensitivity analysis, four elastic net models were fit, which can select groups of correlated features, and may better mimic biological pathways.

Internal replication on ESKD outcome. To evaluate the models on time-to-ESKD, six Cox models were fit, in which predictors were variables selected in the six eGFR slope outcome analyses. Likelihood ratio tests were used to compare each Cox model to its corresponding model of only clinical variables. To quantify model performance, 5-fold cross validation repeated 100 times was used to estimate mean and 95% CI of the C-statistic. Predictors used in these six Cox models, were the predictors selected in the corresponding eGFR slope models; no tuning or variable selection was used in the Cox models. Further training on ESKD was intentionally avoided to assess if predictors of eGFR slope were also predictive of long-term outcomes (i.e., ESKD).

Enrichment analysis. Definitions of well-known biological pathways by their respective involved metabolites were obtained from HMDB [Wishart et al., Nucleic Acids Res. 2018; 46(D1):D608-d17]. 743 pathways were considered and performed a hypergeometric test for each pathway definition via:

$$p - value = f ( x \vert M , K , N ) = \frac{( \begin{matrix}K \\ x\end{matrix} ) ( \begin{matrix}M - K \\ N - x\end{matrix} )}{( \begin{matrix}M \\ N\end{matrix} )} \qquad ( 1 )$$

where M=# of measured metabolite ions, K=# of measured compounds in the pathway definition, N=# of “hits”, i.e., selected/prognostic metabolite ions, and x=# of “hits” that mapped to the pathway. The p-values were corrected for multiple testing using the Benjamini-Hochberg method.

Assay validation. Using a targeted assay, selection and annotation of 15 metabolites detected by untargeted analysis, which were prognostic in the ESKD analysis were validated. Again, Cox models were fit, adjusted for 9 clinical variables. To compare untargeted and targeted analyses, standardized hazard ratios (HR) are presented.

### B. Results

Participant Characteristics. At entry, participants (n=995) were mean 59.9 years, 56% male, 44% white, and 42% black; on average (mean (SD)), they were obese (BMI 34.2 (7.9)), had poor diabetes control (HbA1c 7.6 (1.5)%), and had moderate-to-poor kidney function (eGFR 40.6 (11.2) ml/min/1.73m₂). The eGFR decline (slope) averaged 1.8 (SD=1.9) ml/min/1.73 m²/yr; 36% 137 (N=360) had ESKD during the 10-year study (range: 2-10 years). Subjects with missing clinical variables (<2%) were excluded.

Associations between single metabolites and disease outcomes. Of 698 ions, 89 were significantly correlated with eGFR slopes without adjustment for clinical variables (FDR corrected p<0.05). In adjusted analyses, 6 ions remained significant with β-coefficients from −0.45 to 0.3. Also, 123 (unadjusted) and 99 (adjusted) ions were significantly associated with ESKD in Cox regression. After adjustment, HRs ranged from 1.12 to 1.84. The prognostic ion set contained several amino acids (valine, isoleucine, tryptophan) and other compounds e.g., hydroxybutanoic acid. Only one ion, annotated as 3-(4-Methyl-3-pentenyl) thiophene (Ion Index1098), was associated with both eGFR slope and ESKD in adjusted models with coefficients (95% CI) of −0.44 (−0.68, −0.21) for the eGFR slope, and HR (95% CI) 1.84 (1.45, 2.32), indicating that higher abundance of this compound might be associated with worse DKD progression. Henceforth, the 99 ESKD-151 associated metabolites will be denoted the 99-ESKD-associated set.

Multivariable prognostic metabolites for eGFR slope outcome. Each of the lasso or random forest models selected 9-30 variables resulting in 49 (out of 698) ions across the 6 prognostic models, denoted the 49-eGFR-associated set. Baseline albuminuria, blood pressure, and HbA1c were selected in all 6 models, and unsurprisingly, higher levels of these clinical markers were associated with steeper eGFR decline; race was also selected in all 6 models; 3,4-Dicaffeoyl-1,5-quinolactone, was selected in all models except model 1 (clinical only model). Nine ions (annotated as 3,4-Dicaffeoyl-1,5-quinolactone, Butynal, 3-(4-Methyl-3-pentenyl)thiophene, C10:3, Zalcitibine, Asparaginyl-Hydroxyproline, Valine (or Betaine), Argynil-glutamine, Pentose (or Diisopropyl disulfide) were selected in at least 3 models; an additional 13 ions were selected in at least 2 models. Elastic net models had similar C-statistics, so the more parsimonious lasso models were retained.

Internal Replication. The final selected features from each of the 6 eGFR slope models were tested on the ESKD outcome in Cox models. The likelihood ratio test p-values were <0.0001 when each of the 6 models was compared to the corresponding model with only clinical features, i.e., adding metabolite ions improved model fit significantly. Several ESKD models had similar median C-statistics, ranging from 0.82 to 0.85. The best (model 2) with 29 variables, had cross-validated median (95% CI) C-statistic of 0.85 (0.85, 0.86). This model selected 20 metabolite ions via Lasso; 14 were significantly associated with eGFR slope as evidenced by their bootstrap 95% CIs which excluded 0. Of greater interest are the five ions significantly (5%-level) associated with time-to-ESKD. Higher abundance of valine (or betaine) and 3-(4-methyl-3-pentenyl) thiophene, were each associated with increased risk of ESKD (HR 1.44 and 1.65 respectively); higher asparaginyl-hydroxyproline abundance was associated with lower ESKD risk (HR=0.7). Two other significantly associated compounds were pipezethate and aminophylline. Importantly, since our Lasso model was not trained on ESKD outcome, the Cox model results are expected to be valid and not influenced by model selection. Sensitivity analysis including ACE inhibitor or ARB were ran and use in Cox regression for Model 2. There were negligible changes in the Cox model coefficients for the 29 variables in Model 2; the HR (95% CI) for ACE inhibitor or ARB use was 1.22 (0.92, 1.63, p=0.17), and this not statistically significant at 5% significance level.

Enrichment Analysis. Pathway enrichment was conducted based the 49 eGFR-associated, the 99-ESKD associated, and the combined 49- and 99-sets (=131 ions). Thus in equation (1) M=698; N=49 or 99 or 131; K varied by pathway; and x=# of ions in a pathway and also in the prognostic 49-, 99- or 131-set.

Pathways consistently and significantly enriched across both eGFR slope and ESKD models were enzyme deficiencies, acidurias and acidemias, and those related to amino acid metabolism. Of interest, the valine-leucine-isoleucine degradation pathway involved in insulin resistance, cardiometabolic risk, cardiomyopathy and CKD [Manoli and Venditti, Transl Sci Rare Dis. 2016; 1(2):91-110; White and Newgard, Science. 2019; 363(6427):582-3]; the 2-aminoadipic 2-oxoadipic aciduria and lysine degradation pathways implicated in diabetes and kidney disease [Sell et al., Ann NY Acad Sci. 2008; 1126:205-9; Verzola et al, J Inherit Metab Dis. 2012; 35(6):1011-9]; and the tryptophan pathway [Cheng et al., Sci Rep. 2020; 10(1):12675; de Vries et al., Am J Physiol Renal Physiol. 2017; 313(2):F475-f86; Debnath et al. Int J Tryptophan Res. 2017; 10:1178646917694600] were enriched in the 99-ESKD and combined 131-ion sets.

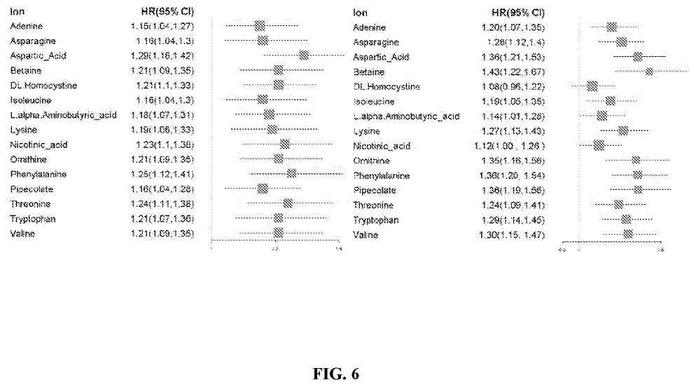

Validation by targeted CE-MS approach. In the targeted validation analysis, 13 of 15 metabolites were significantly associated with ESKD. HR (95% CI) in the untargeted and targeted analysis were comparable, as shown in FIG. 6.

Untargeted Urine metabolomics assay: sample processing and feature extraction. Frozen 24-hour urine samples from 995 CRIC participants and a healthy control were thawed, centrifuged and precipitate-free supernatants were diluted 1:50 in double distilled water in 96 well polypropylene storage microplates (AB-1058, Abgene) sealed with easy-peel heat sealing foil (AB-0745, Abgene) and kept frozen until analysis. Diluted urines were shipped on dry ice and stored at −80 Celsius up to 2 weeks prior to data acquisition by mass spectrometry. Samples were injected in duplicate (i.e., technical replicates) with an MPS 3 xt autosampler (Gerstel) coupled to an Agilent 6550 Q-TOF mass spectrometer (Agilent Technologies) by non-targeted flow injection analysis (Fuhrer et al., 2011). Briefly, the flow rate was 150 μL/min of mobile phase consisting of isopropanol/water (60:40, v/v) buffered with 5 mM ammonium fluoride, and for online mass axis correction, homo-taurine and hexakis (₁H, ₁H, ₃H tetrafluoropropoxy) phosphazine (HP-0921, Agilent Technologies) were added to the mobile phase. Profile mass spectra (MS1) were recorded in 4 Ghz acquisition mode from 50 to 1000 m/z in negative ionization mode with the following source settings: temperature 225° C., drying gas 11 L/min, nebulizer pressure 20 psig, sheath gas temperature 350° C., sheath gas flow 10 L/min, Vcap voltage 3500 V, nozzle voltage 2000 V, fragmentor voltage 350 V and Oct 1 RF Vpp voltage 750V. All steps of data processing and analysis were performed with Matlab R2017b (The Mathworks) using functions embedded in the bioinformatics, statistics, database, and parallel computing toolboxes. After sample alignment and gap-filling, correction for ion intensity drift over time and between plates was performed, and the common mass axis was recalibrated using known frequently occurring ions. Approximately 15k commonly observed ions masses were annotated based on accurate mass comparison using 1 mDa mass tolerance against the Human Metabolome Database HMDBv4.0, resulting in 1899 annotated ion masses assuming single deprotonation.

Targeted Urine metabolomics assay: sample processing and feature extraction. A subset of 15 candidate metabolites from the untargeted panel were assayed by using a quantitative capillary electrophoresis (CE) coupled to mass spectrometry method (ZipChip-Qexactive) to validate metabolite ion identification and annotations from the untargeted method.

For the ZipChip™ assay, 10 μL of urine sample or calibration standard mixture was mixed with 90 μL of extraction solution containing 80% methanol, 100 mM ammonium acetate and 1 μM of stable isotope labeled internal standards in a 96 well plate. After mixing, samples or calibration standards were kept in a −20° C. freezer for at least one hour and then centrifuged at 5000 rpm for 5 minutes. A 50 μL supernatant was transferred to a clean 96 well plate for analysis. Metabolite separation was achieved with a microfluidic chip which integrates capillary electrophoresis (CE) with nano-electrospray ionization through ZipChip™ interface (908 Devices, Boston, MA). For each sample, 20 μL solution was placed into the sample well and subsequently injected into the HS chip using a 10-s load time. A field strength of 1000V/cm was applied for separation over 4 minutes using a background electrolyte solution consisting of 2% formic acid in 50% methanol in water. The mass spectrometry acquisition was done with Q-Exactive mass spectrometer (Thermo, San Jose, CA). The resolving power was 17,500 with an AGC target of 3×10⁶, maximum injection time of 20 ms, and scan range of 75-500 m/z. Thermo Scientific's software Xcalibur-Quan Browser was used for quantitative data processing. Calibration curves for all metabolites were included with each plate. The targeted assay metabolite data were normalized to urine creatinine measured at the CRIC Central Laboratory.

Untargeted metabolomics data: filtering metabolic features. Technical replicate data for each sample were leveraged to develop criteria for filtering out metabolite ions that showed poor reproducibility. A total of 1899 annotated metabolite ions were measured for the 995 CRIC subjects along with a pooled urine sample from the healthy subject, as quality control (QC). All data were collected in duplicates. The three QC sample per plate were randomly run within each 96 well plate in duplicates leading to 6 measurements per plate and a total of 198 measurements across 33 plates. The filtering process consists of three steps. In step one, only the QC data was used. Three reliability metrics, the correlation coefficient between the technical duplicates (Spearman and Pearson QC CC), intraclass correlation (QC ICC) and coefficient of variation (QC CV) for each metabolite ion abundance were calculated. The QC ICC for each metabolite ion abundance was calculated as the variance between technical duplicates divided by the total variation (including batch and replicate variation); the QC CV was calculated as the standard deviation between technical duplicate abundance divided by the mean of the samples. Low correlations, high ICC or high CV values would indicate poor reliability, i.e., high variability relative to signal. We formalized these criteria and excluded ions for which any of the following was true:

$$QC\_Spearman < 0.85 or QC\_Pearson < 0.85$$

$$QC\_CV \geq 0.05$$

$$QC\_ICC \geq 0.05$$

These cut offs are based on standard assay reliability metrics. A filtering step two was implemented using the CRIC samples. Here the objective was to filter out metabolite ions which exhibited low biological variability. To this end an intraclass correlation (CRIC ICC), as the ratio of between subject to total sample variation was calculated; ions with ICC<=0.35 were further excluded.

Thus, in summary, the final set of 698 retained metabolite ions satisfied all criteria: QC Spearman>=0.85 and QC_Pearson>=0.85 and QC_CV<0.05 and QC_ICC<0.05 and CRIC_ICC>0.35. The technical replicates of the metabolite ion abundances (two per sample) were then averaged. In the final filtering step, metabolite ions with low abundance were excluded to exclude non-informative ions in the noise range. The ions that passed the filtering criteria and count threshold, constituted the final metabolite ion feature set for modeling.

Targeted metabolomics data: variability. The QC CV of metabolomic data is calculated same way as described above. Inter-day CV which was obtained from health human urine samples was calculated. Of the 15 metabolites included in the target analysis, 7 of them have a QC CV of <0.05, 3 of them have a QC CV of between 5% and 10%. 4 of them, Ornithine, Isoleucine, Asparagine, Betaine, have a QC CV of more than 10%.

High-throughput metabolomics offers great potential, yet the scale and measurement errors, raise analytic and inferential challenges. To address these challenges, two steps are implemented: (1) identify a sparse prognostic set of metabolites after adjusting for known clinical factors, (2) use pathway analysis to elicit biological meaning. First, using an untargeted assay, we tested single metabolite associations with eGFR slope and ESKD.

As a next step, we conducted a quantitative targeted assay of 15 amino acid metabolism-metabolites from the 99 (untargeted) ions, and validated the prognostic value of 13 (of 15). Our finding of the prognostic value of urine amino acids in the large CRIC cohort even after adjustment for clinical factors, suggests that additional amino aciduria, independent of albuminuria, may be risk factor for DKD progression.

Using multivariable analysis, asparginyl-hydroxyproline and arginyl glutamine are identified as novel findings in DKD research. In addition, valine, a glucogenic branched chain amino acid, and C10:3 (an acyl-carnitine) and 3-(4-Methyl-3-pentenyl) thiophene were also identified. Importantly, in the multiple adjusted Cox model with highest C-statistic, we identified several interesting prognostic (p<0.05) metabolite ions. Valine (or betaine) and 3-(4-Methyl-3-pentenyl) thiophene were associated with 44% and 65% higher hazard of ESKD, respectively, per doubling of ion abundance. Importantly, the Cox model analysis, albeit on the same cohort, did not involve any training or variable selection, hence HRs are likely less biased.

To systematically leverage the biological content of prognostic metabolite ions, we conducted enrichment analysis, using pathway, disease and function definitions from the HMDB. We focused on metabolic pathway enrichment, since pathway definitions are less prone to curation anomalies, more finite and well-defined compared to disease and function definitions. We identified branched chain amino acid pathways, namely valine-leucine-isoleucine degradation, isovaleric acidemia/aciduria, propionic acidemia and methylmalonic acidemia. Our study found that higher levels of tryptophan were associated with higher ESKD risk in both untargeted and targeted analyses, and the tryptophan pathway was significantly enriched in our set of ESKD-related metabolites. To our knowledge these findings are novel, and further investigation of urine tryptophan is warranted.

In summary, large-scale untargeted and targeted metabolomics analysis were conducted using a diverse cohort and identified several metabolites implicated in DKD. We used robust statistical models incorporating rigorous techniques to reduce overfitting: cross-validation for feature selection, replicating results on a long-term clinical outcome, and validation using a targeted assay. While we highlighted and validated a few prognostic metabolites and related pathways, the most important aspect of our work is that we identified 131 potentially prognostic metabolites in our untargeted analysis, several of which are not addressed before. We hope that providing this list will spur further investigation of these promising metabolites and enhance understanding of DKD, eventually leading to better treatments and disease management.

### Example 2

### Role of Adenine for Kidney Failure and Mortality in Diabetes

Progression of diabetic kidney disease and CKD remains an enigma. This example uses an unbiased multi-omic approach with a focus on spatial metabolomics on human kidney biopsies via the Kidney Precision Medicine Project. A major pathway associated with tubulointerstitial disease was the polyamine/purine pathway and one potential critical metabolite that is connected with both pathways is adenine. MALDI-mass spec imaging with optical overlay localized adenine to regions of atrophic tubules, arteriosclerotic blood vessels and sclerotic glomeruli in kidney biopsies from patients with diabetic and non-diabetic chronic kidney disease. In a rat model of diabetic kidney disease (ZDF rat), tissue adenine and urine adenine/creatinine correlated with glomerulosclerosis and tubulointerstitial fibrosis. In a Pima Native American cohort with diabetes that underwent protocol renal biopsy after urine measurements, the urine adenine/creatinine levels were predictive of glomerulosclerosis (r=0.57, p=0.03, n=15) and performed better than urine ACR (r=0.22, p=0.17). Further, the upper tertile of urine adenine/creatinine ratio was prognostic of measured GFR decline in Pima Native Americans with normoalbuminuria (HR 4.3, CI 1.14-16.21, p=0.03, n=32). This pattern was consistent in patients with diabetes in the Chronic Renal Insufficiency Cohort study (CRIC), as the upper tertile of urine adenine/creatinine ratio was found to be prognostic for kidney failure in patients with diabetes and normo-albuminuria (top tertile HR 4.42 CI 1.56, 12.5, p=0.005, n=302) and middle tertile (HR 3.01, CI 1.09-8.32, p=0.034, n=301) as compared to the lowest tertile (n=301). Thus, urine adenine is a new prognostic biomarker for ESKD in diabetic kidney disease with normoalbuminuria.

### A. Results

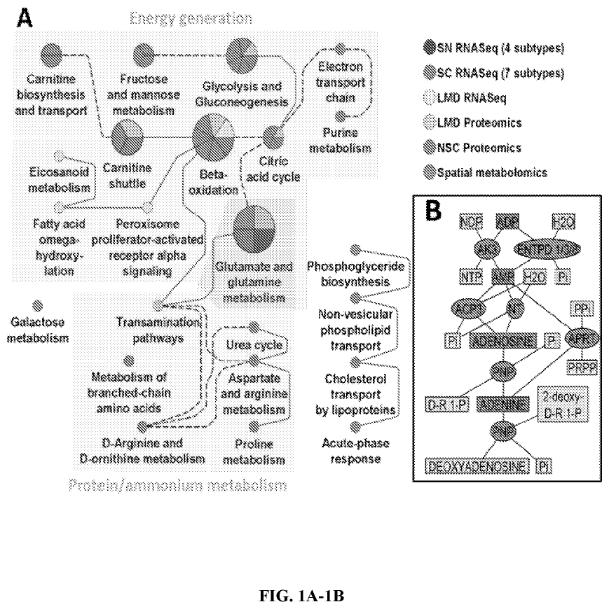

Higher level bioinformatic directed data integration across KPMP TISs. Integrative analysis of multiple omic layers from different TISs was demonstrated to provide more robust, interpretable, and translatable data compared to any single omics layer alone. An integrative approach centered around metabolic pathways by creating a map of all biochemical reactions and metabolic pathways that interface with other subcellular processes including transport systems, signaling pathways, and cytoskeletal systems were developed. Second, for six experimental technologies within KPMP, overlapping, complementary, and interdependent pathways and reactions within these cellular and subcellular processes were predicted. Here, spatial metabolomics, single-cell transcriptomics, single-nucleus transcriptomics, laser microdissected (LMD) transcriptomics, as well as LMD and near-single-cell proteomics applied to KPMP samples and additional human kidney samples used to build the reference tissue atlas were considered. Using pathway enrichment analysis, consistent results were documented arising from the identification of overlapping genes, proteins and metabolites which led to identifying biochemical pathways part of the same higher-level function. The spatial metabolomics data served as anchors for such biochemical pathway integration as it provides the end-product of metabolic pathways. The integrated functional relationships predicted interacting pathways such as beta-oxidation, carnitine shuttle, and carnitine biosynthesis within tubular compartments (FIG. 1A) and helped to define new proximal tubule segments based on bioenergetic profiles. In addition, tubulointerstitial metabolites indicated a strong functional relationship between purine metabolism and glycolysis. Further, the nephrotoxic metabolite adenine was identified to be a potential key metabolite for the tubulointerstitial compartment from the integrated bioinformatic analysis (FIG. 1B). As described above in Example 1, urine adenine was identified to be associated with ESKD in an untargeted urine metabolomic analysis of 995 patients with diabetes in the CRIC study. Therefore, a further in-depth analysis of adenine was undertaken using spatial metabolomics.

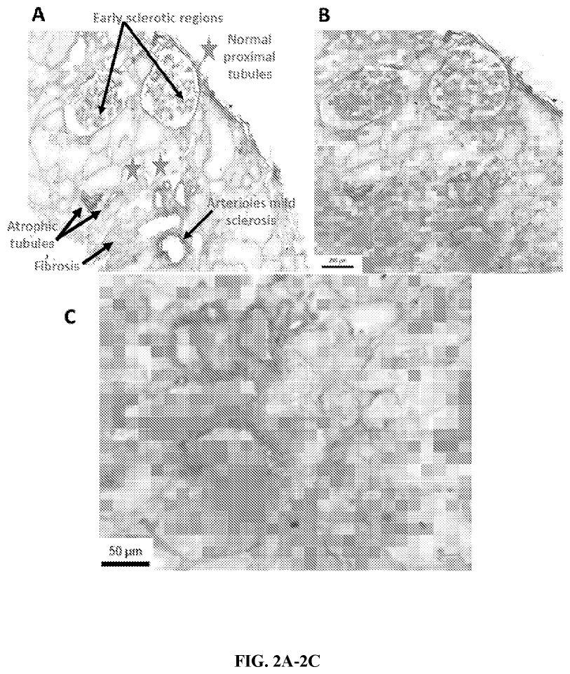

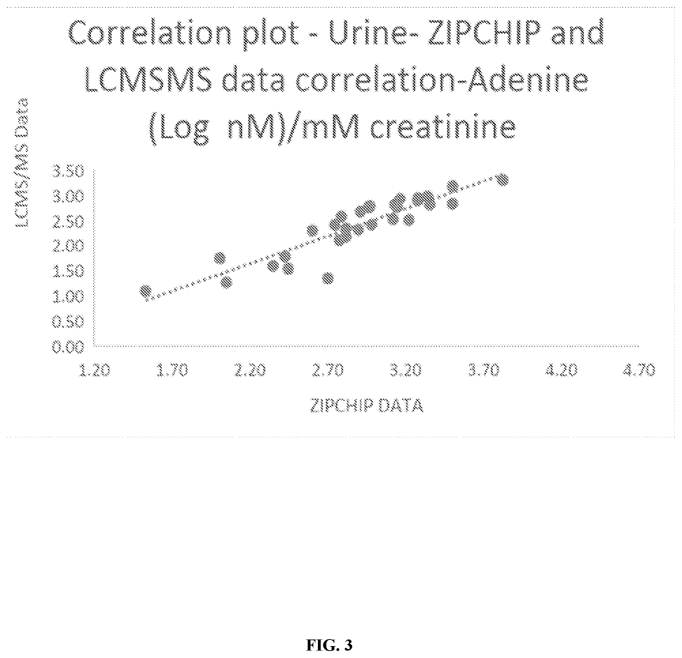

Spatial metabolomics localizes adenine with glomerulosclerosis, arteriosclerosis and tubular atrophy. Spatial metabolomics overlaid with histology studies of selected human kidney tissue with pathologic features were interrogated for adenine. There was a clear localization of adenine in the glomeruli and tubulointerstitial compartments (FIG. 2A and FIG. 2B). High power resolution of adenine localized adenine adjacent to atrophic tubules and a region of tubule-interstitial fibrosis (FIG. 2C). Adenine also localized to the vascular compartment in the peritubular space as the vascular wall in small vessels with arteriosclerosis (FIG. 2C). Given the role of adenine in tissue samples, a targeted high throughput urine adenine assay was developed and found excellent correlation with spatial adenine in tissue sections (r=0.73, p<0.001, Table 1) and LC-MS/MS analysis of urine adenine/creatinine (r=0.90, p<0.001, FIG. 3).

TABLE 1
Baseline clinical characteristics of Pima Native Americans
prior to kidney biopsy. Samples from patients who were not
on renin-angiotensin inhibitors were evaluated for urine
adenine levels at two time points before kidney biopsy.

| | Mean (SD)/Median (IQR)/ | |
|---|---|---|
| | frequency (n = 15) | |
| Age (years) | 41.8 ± 10.3 | |
| Male sex (%) | 3 | (20%) |
| Diabetes duration (years) | 13.2 ± 4.7 | |
| HbA1c (%) | 9.6 ± 2.2 | |
| MAP (mmHg) | 92.1 ± 7.4 | |
| BMI (kg/m²) | 34.2 ± 7.4 | |
| Glomerular filtration rate (ml/min) | 141 ± 52 | |
| ACR (mg/g) | 26 | (15-333) |
| Urine adenine as fraction of initial measure | 1.41 | (0.83-4.18) |

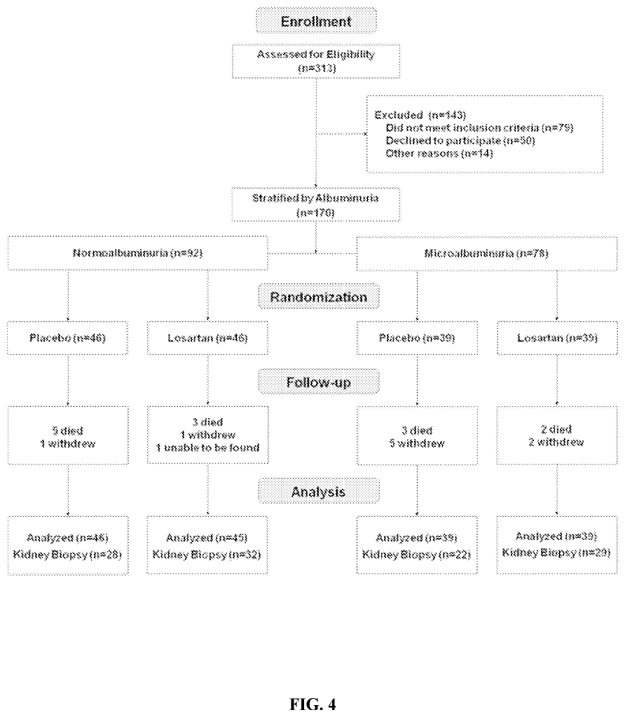

Urine adenine in PIMA Native American population correlates with kidney pathology and clinical outcomes. Given the co-localization of spatial adenine with pathologic features, the non-invasive urine adenine levels measured prior to a kidney biopsy were evaluated as a predictor to glomerular pathology. In collaboration with the PIMA Native American study (PMID 29305527) (described in FIG. 4), urine adenine was measured at two specified time intervals before a research protocol biopsy in 15 patients not placed on renin-angiotensin inhibitor therapy. The baseline clinical characteristics are described in Table 1. By assessing the change in urine adenine over baseline with the pathology score, there was a statistically significant correlation with the change in urine adenine and glomerulosclerosis (see Table 2). The correlation between urine adenine and % glomerulosclerosis remained significant even after adjusting for urine ACR (r=0.650, p=0.007). Surprisingly, the change in urine ACR prior to the renal biopsy was not statistically correlated with the degree of glomerulosclerosis (r=0.116, p=0.65, Table 2). There was a trend of negative correlation of urine adenine with percentage of fenestrated endothelium and a positive correlation with cortical interstitial volume.

TABLE 2
Urine Ad/Cr at 6 m-1 y prior to kidney biopsy predicts
Glomerulosclerosis in Pima Native Americans (n = 15)

| | | Cortical |
|---|---|---|
| Urine Biomarker | | Interstitial Fractional |
| in Pima group | GS (by PAS) | Volume |
| Urine Albumin/Creatinine ACR | 0.22, p = 0.17 | 0.057, p = 0.83 |
| Urine Ad/creatinine | 0.57, p = 0.03 | 0.38, p = 0.16 |

Urine adenine correlates with decline in GFR To determine if the urine adenine levels had clinical predictive value, the cohort of 60 patients who had urine adenine levels measured were divided into tertiles (t1 3-67 nM adenine/mM creatinine (4-80 mg adenine/gram creatinine); t2 72-411 nM/mM (86-491 mg/gm); t3 429-2954 (512-3529 mg/gm)) and correlated with the iothalamate-based measured GFR change (Table 3). The outcome evaluated was >=40% decline in GFR over a median follow-up of 7.6 years (IQR 3.8-11.8). 47 of the 60 participants met this outcome criteria. The urine adenine levels correlated with measured GFR decline in patients with normoalbuminuria (HR=4.3, 95% CI 1.14-16.21, p=0.03) when comparing tertile 3 (highest urine adenine/creatinine group) vs tertile 1. There was a trend toward significance in the normoalbuminuria group between tertile 2 vs tertile 1, but did not reach statistical significance (HR=2.3, 95% CI 0.62-8.56, p=0.21). In the microalbuminuria group there was a significant relationship between tertile 2 and tertile 1 (HR=4.96, 95% CI 1.10-22.34, p=0.04). The urine adenine/creatinine ratio did not add to the predictive value in the macroalbuminuria group.

TABLE 3
Adjusted Hazard Ratios of >=40% decline in GFR between tertiles of urine adenine/creatinine
in normoalbuminuria, microalbuminuria and macroalbuminuric patients in the Pima Native American study.

| | | T2 vs T1 | | | T3 vs T1 | | |
|---|---|---|---|---|---|---|---|
| PIMA Native Americans | N | HRR | 95% CI | P value | HRR | 95% CI | P value |
| Normoalbuminuria | 32 | 2.31 | 0.62-8.56 | 0.21 | 4.30 | 1.14-16.21 | 0.03 |
| Microalbuminuria | 16 | 4.96 | 1.10-22.34 | 0.04 | 2.93 | 0.69-12.42 | 0.15 |
| Macroalbuminuria | 12 | 0.54 | 0.09-3.13 | 0.49 | 0.75 | 0.18-3.13 | 0.69 |
| All | 60 | 1.55 | 0.74-3.25 | 0.25 | 2.46 | 1.15-5.220.02 | |

Urine adenine identifies patients with future development of ESKD in the CRIC cohort. As the data from the PIMA Indian population supported the role of urine adenine as a marker of kidney pathology in patients and indicated a role in kidney disease progression, we evaluated a large, well characterized cohort of patients with diabetic kidney disease from the Chronic Renal Insufficiency Cohort (CRiC). Baseline urine adenine was measured from samples at the time of entry into CRIC and patients had yearly follow-up for evaluation of need for ESKD outcomes (eGFR<15 ml/min/m2, or kidney replacement therapy). The baseline clinical characteristics are shown in Table 4. Of the 995 subjects evaluated 292 had normoalbuminuria (ACR ≤30 mg/g creatinine). The group of subjects with a urine adenine/creatinine ratio in the lowest tertile was used as the reference group in the analysis of the association between the ESKD outcome and urine adenine/creatinine ratio.

TABLE 4
Baseline characteristics of patients with diabetes in the CRIC
study. BMI, body mass index; eGFR, estimated glomerular filtration
rate; HbA1c, hemoglobin A1c; UACR, urine albumin-to-creatinine
ratio; *Continuous ACR is summarized using median (IQR, interquartile
range) because of its skewed distribution. All other continuous
variables are summarized using mean ± SD.

| Clinical Variable | Mean ± SD/N (%) | |
|---|---|---|
| Age (years) | 59.9 ± 9.4 | |
| Sex | | |
| Male | 561 | (56) |
| Female | 434 | (44) |
| Race | | |
| White | 440 | (44) |
| Black | 420 | (42) |
| Other | 135 | (14) |
| Smoked 100 Cigarettes | | |
| Yes | 565 | (57) |
| No | 430 | (43) |
| BMI (kg/m²) | 34.2 ± 7.9 | |
| Mean Arterial Pressure (mmHg) | 90.0 ± 13.4 | |
| eGFR (ml/min/1.73²) | 40.6 ± 11.2 | |
| HbAlc (%) | 7.6 ± 1.5 | |
| ACR (mg/g) | | |
| ≤30 | 292 | (30) |
| 30-300 | 272 | (28) |
| ≥300 | 420 | (43) |
| Continuous ACR\hat{ } (mg/day) Median | 0.16 | (0.92) |
| (IQR) | | |

After adjustment for differences in clinical variables, the upper two s of urine adenine within the normoalbuminuria group had a strong effect size for ESKD outcomes (Table 5). The upper tertile of urine adenine had a hazard ratio 4.42 (CI 1.56. 12.5, p=0.005) and the middle tertile had a hazard ratio of 3.01 (CI 1.09-8.32, p=0.034) as compared to the lowest tertile (Table 5). The upper two tertiles did not have a significant increased risk in the microalbuminuria group (Table 5).

TABLE 5
Adjusted Hazard Ratio for ESKD Outcome based on tertiles of urine adenine
in the normoalbuminuric and microalbuminuria group from the CRIC Study.
The analyses were adjusted for baseline age, sex, eGFR, mean arterial
blood pressure, hemoglobin A1C and duration of diabetes.

| | | T2 vs T1 | | | T3 vs T1 | | |
|---|---|---|---|---|---|---|---|
| CRIC patients | N | HRR | 95% CI | P value | HRR | 95% CI | P value |
| Normoalbuminuria | 292 | 3.01 | 1.09-8.32 | 0.034 | 4.42 | 1.56-12.5 | 0.005 |
| Microalbuminuria | 272 | 1.20 | 0.71-2.02 | 0.5 | 1.52 | 0.86-2.69 | 0.2 |

Spatial adenine and urine adenine correlates with kidney pathology in ZDF rat model of diabetic nephropathy. As chronological assessment of a marker with pathology is difficult to establish in patients, a pre-clinical animal model was evaluated. Spatial metabolomics was performed in the ZDF rat model and spatial adenine was correlated with glomerulosclerosis (Table 6). Interestingly, glomerular adenine had a poor but significant correlation with glomerulosclerosis, however non-glomerular and overall whole slide image (WST) adenine was much better correlated with glomerulosclerosis in the ZDF rat at 6 months of age.

TABLE 6
Correlation of spatial adenine with glomerulosclerosis

| MALDI-MSI of ZDF Kidney | Correlation with Glomulerosclerosis |
|---|---|
| Glom Metabolite Adenine | R = 0.04, p < 0.01 |
| Non-Glom Metabolite Adenine | R = 0.44, p < 0.01 |
| Whole Slide Image Metabolite X | R = 0.54, p = 0.03 |

A non-invasive urine assay was developed using a high throughput ZipChip based mass spec based approach and found to be highly correlated with LC-MS/MS measurement of urine adenine (r2=0.82, p value, n=23 samples, FIG. 3).

The urine assay was also found to highly correlate with glomerular, non-glomerular and WSI levels of adenine indicating that urine adenine would reflect kidney levels of adenine (Table 7).

TABLE 7
Spatial adenine correlated with urine adenine in ZDF rat model.

| MALDI-MSI | Correlation with Urine Adenine Assay |
|---|---|
| Glom Adenine | R = 0.64, p < 0.001 |
| Non-Glom Adenine | R = 0.81, p < 0.001 |
| Whole Slide Image Adenine | R = 0.73, p < 0.001 |

The urine adenine assay was then evaluated in a long term 9m model of diabetic nephropathy in the ZDF rat. Urine levels measured at 30 weeks of age predicted both glomerulosclerosis and tubulointerstitial disease assessed at 9m of age. Surprisingly the urine adenine performed better to predict glomerular and tubulointerstitial pathology than urine ACR (Table 8).

TABLE 8
Urine adenine and urine albumin levels
at 30 weeks of age associated with glomerulosclerosis
and tubulointerstitial disease at 36 weeks of age.
Urine Ad/Cr at 30 weeks predicts Glomerular
and Tubulointerstitial Pathology in ZDF rat

| | | TIF (tubular |
|---|---|---|
| Urine Biomarker | | atrophy and |
| in ZDF- | | interstitial |
| vehicle group | GS (by PAS at 36W) | fibrosis score) |
| Urine Albumin/Creatinine | 0.21, p < 0.001 | 0.04, p < 0.001 |
| ACR (measured at 30W) | | |
| Urine X/create | 0.45, p < 0.001 | 0.31, p < 0.001 |
| (measured at 30W) | | |

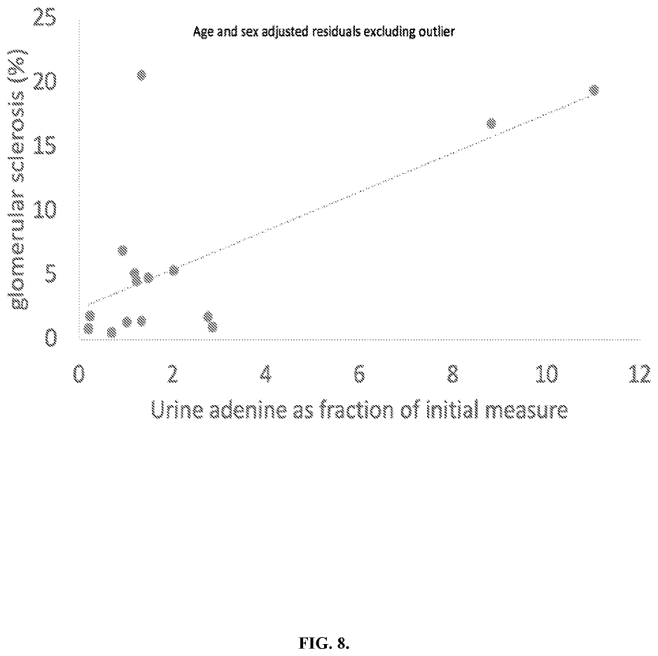

Urine adenine in PIMA Indian population demonstrates that urine adenine correlates with kidney pathology and clinical outcomes. Given the strong correlation of urine adenine with glomerulosclerosis in the ZDF model, we evaluated whether urine adenine levels performed prior to a kidney biopsy may predict glomerular pathology. In collaboration with the PIMA native American study group, urine adenine was measured at two specified time intervals before a research protocol biopsy in 15 patients not placed on renin-angiotensin inhibitor therapy (as part of a prior randomized study). By assessing the change in urine adenine over baseline with the pathology score, there was a surprising strong correlation with the change in urine adenine and glomerulosclerosis (see FIG. 8 and Table 9).

The correlation between urine adenine and % glomerulosclerosis remained significant even after adjusting for urine ACR (r=0.650, p=0.00⁷). Surprisingly, the urine ACR prior to the renal biopsy was not statistically correlated with the degree of glomerulosclerosis (r=0.116, p=0.65).

The cohort of 60 patients who had urine adenine levels measured were divided into tertiles (t1 3-67; t2 72-411; t3 429-2954). The outcome is >=40% decline in GFR (using our standard imputation technique) with a median follow-up of 7.6 years (IQR 3.8-11.8). We have 47 cases for the 60 participants included in the analyses. The urine adenine levels correlated with measured GFR decline in patients with normoalbuminuria (HR=4.3, 95% CI 1.14-16.21, p=0.03, n=32). Of note, the GFR was measured with inulin based measurements and is thus a measure of true GFR.

TABLE 9
Association of urine adenine with kidney pathology features

| | Univariate | | Age & sex | |
|---|---|---|---|---|
| | r | P | r | P |
| GBM width (nm) | 0.096 | 0.72 | 0.080 | 0.77 |
| Mesangial fractional volume | −0.063 | 0.82 | 0.009 | 0.99 |
| Surface density (um²/um³) | −0.074 | 0.78 | −0.039 | 0.89 |
| Cortical interstitial fractional volume | 0.312 | 0.24 | 0.310 | 0.24 |
| Mean glomerular volume (10⁶ um³) | −0.343 | 0.19 | −0.376 | 0.15 |
| Percentage glomerular sclerosis (%) | 0.502 | 0.047 | 0.673 | 0.004 |
| Foot process width (nm) | 0.430 | 0.10 | 0.392 | 0.13 |
| Podocytes per glomerulus (n) | −0.190 | 0.48 | −0.244 | 0.36 |
| Percentage fenestrated endothelium % | −0.307 | 0.25 | −0.470 | 0.07 |
| Percentage denuded endothelium (%) | 0.339 | 0.20 | 0.379 | 0.15 |
| Total filtration surface (10⁵ um²) | −0.190 | 0.48 | −0.282 | 0.29 |

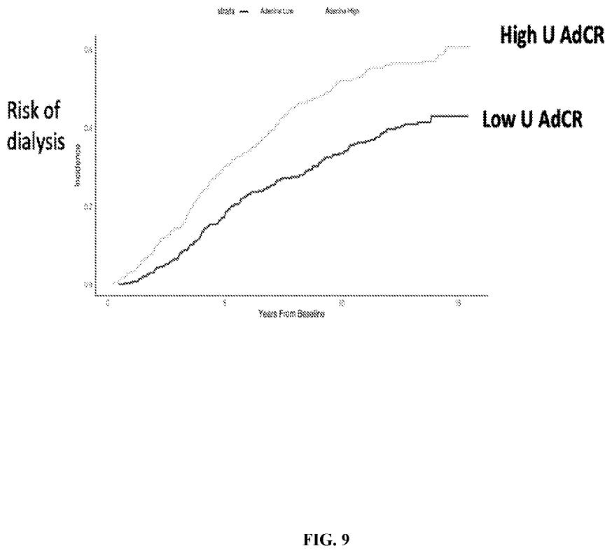

A recent analysis of urine untargeted metabolomics identified urine adenine and several other metabolites along the polyamine pathway (ornithine) to be linked to progression of kidney disease (recent manuscripts and new analysis) indicating that urine adenine could be a risk factor for progression. A targeted assay identified that urine adenine correlated with progression of renal function decline and risk of ESKD. Indeed, a high urine adenine (upper half of levels, give absolute value) indicated a 60% higher risk of developing ESKD than those in the lower half (FIG. 9).

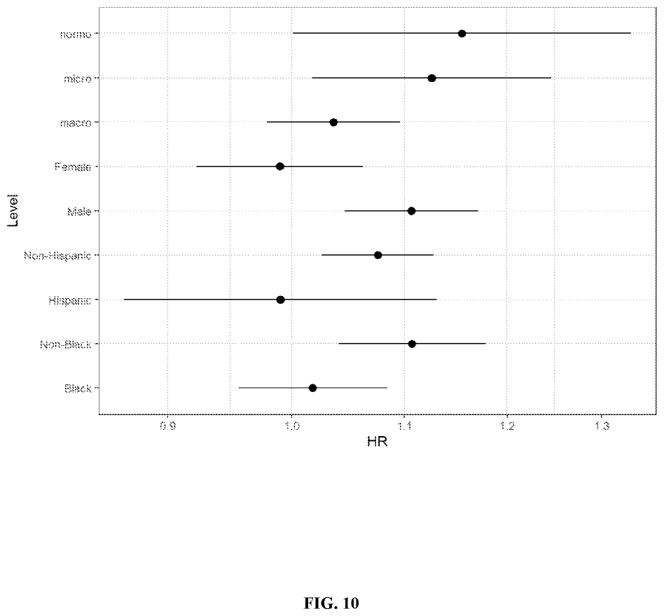

Further sub-group analysis identified that males with elevated urine Ad/cr had a HR (1.11) and those with micro- (HR 1.13) or macro-albuminuria had the highest HR (1.15) for risk of progression to ESKD, even after adjustment relevant clinical variables. See FIG. 10.

### B. Methods

Chemicals and Reagents. 1,5-Diaminonaphthaalene (97%) was purchased from Sigma-Aldrich. Indium tin oxide (ITO) glass slides required for MALDI MSI were purchased from Bruker Daltonics (Bremen, Germany) and superfrost plus microscope glass slides essential for PAS and immune staining attained from Fisher Scientific, PA, USA. Alcoholic formalin, distilled, periodic acid and Schiff's reagent from Merck, Darmstadt, Germany. Mayer's Hematoxylin from NovaUltra™. TBS, Ethanol and xylene mounting media and all other reagents were sourced from Sigma-Aldrich (St. Louis, MO) unless otherwise noted.

Sample collection. Human samples were obtained from KPKP and University of Texas Health Science Center, San Antonio (UTHSCSA) institutional review board has been approved all the experimental procedures (IRB No: HSC2017035H). Written consent was attained from all patients. Tissue specimens were obtained by surgical resection in the Department of Pathology, UTH San Antonio. Instantly samples were frozen in liquid nitrogen (without PBS pre-wetting) and stored at −80° C. bio-freezer until analysis.

Tissue Sectioning. A Leica CM1950 cryomicrotome (Leica, Biffalo Grove, IL) was pre-equilibrated to a chamber and blade temperature of −15° C. and −20° C., respectively. Tissues were embedded on ice made with few drops of milli ‘Q’ water. All tissues were sectioned at 10 μm thickness and thaw mounted onto indium tin oxide (ITO) coated glass slides for MSI and 4 μm thickness sections thaw mounted onto microscopy glass slides for PAS and immunofluorescence analysis. The glass slides to be analyzed for MALDI were placed into a vacuum desiccator for approximately 30 min before matrix application, remaining slides were stored at −80° C. bio-freezer until further analysis.

Optical Imaging. A multimodal imaging approach was employed for investigating regional localization of glomeruli, proximal and distal tubule in the tissue section. In this approach, optical microscopy is integrated with MSI. Bright-field microscopy visualized the shape of the glomeruli, autofluorescence microscopy outlined the position of glomeruli regions, and PAS staining revealed the localization of glomeruli in the tissue. MSI employing MALDI-MSI visualized the distribution of intact small metabolite ions. All modalities were combined through the presence of fiducial markers visible in all imaging modalities.

Optical images (Auto flourescence (AF), bright filed (BF) and PAS images) are acquired on a confocal microscope (Leica TCS SP8) or Aperio ScanScopeXT (Leica Biosystems). For AF and BF images, images were captured before (pre-AF/BF) MALDI-MSI. Briefly, before the matrix application, the pre-AF/BF images were captured using 10× objective on Leica TSC SP8 confocal microscope at 495 nm-720 nm, 404-488 nm wavelengths and bright field channels will be used for AF/BF imaging. Consecutive serial sections will be obtained for PAS and for immunofluorescent analysis with 3-5 μM frozen sections. The PAS stained slide will be scanned using an Aperio CS2 image capture device with a 40× magnification. The optical image will be upload to METASPACE and SCiLS Lab software for overlay with metabolite images. Data from regions of interests (ROT) (e.g., glomeruli, proximal tubules, distal tubules, interstitium) will be extracted for further analysis.

Matrix application for small metabolite molecules imaging. The DAN-HCl matrix solution was sprayed on ITO glass slide using an HTX™-sprayer (HTX Technologies, Chapel Hill, NC) using the following settings: 75° C. nozzle temperature, 16 passes, 0.060 mL/min flow rate, 1200 mm/min velocity, 2 mm track spacing, 10 psi pressure, 3 L/min gas flow, 20 s dry time between passes, using the “CC” coverage pattern.

Mass Spectrometry Imaging. Thermo Scientific Q Exactive HF-X hybrid quadrupole-Orbitrap mass spectrometer in combination (Thermo Scientific, USA) with a novel elevated pressure MALDI/ESI interface (Spectroglyph LLC, Kennewick, WA, USA) (Belov et al., 2017) was used to obtain MSI images. This modified MALDI-Orbitrap mass spectrometer equipped with a 349 nm, 120 uJ, 5 kHz Nd:YLF laser (Spectra Physics, Mountain View, CA). The 349 nm laser was operated laser repetition rate at 1000 Hz (was 500 Hz) with a laser pulse energy of 2.34 μJ (from 2.18 μJ) in a 5 ns pulse. In IMS experiments, in order to obtain negative ions, a sample was placed on a coordinate table 5 cm from the ion funnel. Produced ions were captured by the ion funnel and transferred to a Q Exactive Orbitrap mass spectrometer (Thermo). Mass spectra were attained in the mass range of m/z 60-900. The Spectroglyph MALDI Injector Software used to control the raster step size on the tissue region to be imaged. To generate images, the spectra were collected at 20-30 μm intervals in both the x and y dimensions across the surface of the sample. The MSI data were recorded using an Orbitrap instrument operating with a nominal mass resolution of 120,000 (from 100,000), a 200 ms ion injection time (was 180 ms injection time), and automatic gain control turned off. Ion images were generated from raw files (obtained from Orbitrap tune software) and position files (obtained from MALDI Injector Software) by Image Insight software (Spectroglyph LLC). The centroid and profile data were exported into the imzML format using Image Insight and SCiLS Lab software version 2019c Pro (SCiLS, Bremen, Germany) separately. Details of general workflow presented below in FIG. 2.

PAS Staining. Fresh frozen biopsy sections were stained with PAS and hematoxylin (Merck, Darmstadt, Germany) to show sclerotic areas and histology of the glomeruli. The following staining procedure from Department of Pathology and Laboratory Medicine, UTHSCSA, San Antonio was used, 4 μm in thickness human kidney tissue sections were mounted onto precleaned superfrost plus microscope glass slides (Fisher Scientific, PA, USA) and stored at −80° C. (see Tissue Sectioning section). The slides were taken out from bio-freezer and dried in desiccator for 30 min before PAS staining procedure. The glass slides were immersed for 10 min into 95% alcoholic formalin than rinse the slides in distilled water 4 times. The glass slides were placed in 0.5% periodic acid solution for 5 min following rinsing with tape water 4 times. For 15 min the glass slides were immersed in Schiff's reagent solution and rinsed in warm tap water for 5 times. The glass slides were then immersed in Mayer's Hematoxylin for 3 min and rinsed in TBS and distilled water 4 times. The glass slides were dehydrated sequentially in 95% alcohol, 100% alcohol, and xylene for 5 seconds in each solution, then mounted and cover slipped. The slides were scanned with Aperio VERSA 200 scanner (Leica Biosystems, IL, USA).

Statistical Analysis. A multivariate analysis of the datasets was obtained from the MSI datasets of human kidney cortex tissue samples. First, the principal component analysis (PCA), as an unsupervised statistical method, was used to detect outliers and find the structure of the data. Next, orthogonal projection to latent structures-discriminant analysis (PLS-DA) as a supervised statistical method was performed to expound diacritic metabolites between two groups. The quality of the PLS-DA model was evaluated by R²X, R²Y and Q², where R² and Q² are the goodness of fit and goodness of prediction, respectively. Metabolite identification and pathway analysis was done to for the annotations extracted from HMDB database in METESPACE using MetaboAnalyst 4.0 (URL metaboanalyst.ca/) was used for metabolic pathways analysis (MPA). The most related pathways were reported based on the p values with the false discovery rate (FDR) less than 0.05.

Urine metabolomics (Zip-Chip™ Analysis). Adenine of plasma samples was analyzed using ZipChip™ coupled with mass spectrometry. Briefly, 10 μL of sample or calibration standard mixture was mixed with 90 μL of extraction solution containing 80% methanol, 100 mM ammonium acetate and 1.5 μM of stable isotope labeled internal standard in a 96 well plate. After mixing, samples or calibration standards were kept in an −20° C. freezer for at least one hour and then centrifuged at 5000 rpm for 10 minutes. A 50 μL supernatant was transferred to a clean 96 will plate for analysis. Metabolites separation was achieved with a microfluidic chip which integrates capillary electrophoresis (CE) with nano-electrospray ionization through ZipChip™ interface (908 Devices, Boston, MA). For each sample, 20 μL solution was placed into the sample well and subsequently injected into the HS chip using a 10-s load time. A field strength of 1000V/cm was applied for separation over 4 minutes using a background electrolyte solution consisting of 2% formic acid in 50% methanol in water. The mass spectrometry acquisition was done with Q-Exactive™ mass spectrometer (Thermo, San Jose, CA). The resolving power was 17,500 with an AGC target of 3×10⁶, maximum injection time of 20 ms, and scan range of 75-500 m/z. Thermo Scientific's software Xcalibur-Quan Browser was used for quantitative data processing.

Bioinformatic and systems medicine analysis. Single cell, single nucleus, laser-microdissected (LMD) transcriptomics, LMD proteomics, Near Single Cell (NSC) Proteomics and spatial metabolomics datasets generated from unaffected tissue in kidney nephrectomy and biopsy samples were obtained from Hansen et al., (URL doi.org/10.1101/2020.07.23.216507), Lake et al (PMID: 31249312) and Menon et al (PMID: 32107344). In these studies, marker genes and proteins were obtained by comparing gene expression in each cell type, subtype and subsegment versus all other cell types, subtypes or subsegments. Marker genes and proteins were removed that did not fulfill the significance criteria (maximum adjusted p-value of 0.05 for single cell and nucleus transcriptomics, maximum p-value of 0.05 for LMD transcriptomics, LMD proteomics and NSC proteomics) and ranked the remaining genes and proteins by significance. The top 500 ranked genes and proteins as well as the tubulo-interstitial metabolites were mapped to enzymes and regulatory proteins involved in polyamine metabolism (PMID: 30181570).

Pima Native American study. All patients from this study were enrolled in a randomized clinical trial (Weil et al. 2013) testing the renoprotective efficacy of losartan vs. placebo in early diabetic kidney disease (ClinicalTrials.gov number, NCT00340678). Glomerular filtration rate (GFR) was measured annually throughout the trial by the urinary clearance of iothalamate. At the end of the six-year clinical trial, participants underwent percutaneous kidney biopsy to determine whether treatment was associated with preservation of kidney structure. Stored urine samples were available for 62 participants prior to the kidney biopsy from the clinical trial who also had biopsy data available. The clinical trial was approved by the Institutional Review Board of the National Institute of Diabetes and Digestive and Kidney Diseases. Each participant signed an informed consent document. For the present study analysis was restricted to participants randomized to placebo who were not taking any renin angiotensin system blocker at the time of biopsy or at the time of collection of either urine specimen used for measurement of urine adenine concentration.

Kidney morphometry. Digital images from the kidney biopsy tissue sections were collected and the kidney structural parameters were quantified by morphometric methods as described previously (Weil et al. 2013). Parameters measured using electron microscopy included glomerular basement membrane width, mesangial fractional volume per glomerulus, glomerular filtration surface density, number of podocytes per glomerulus, podocyte foot process width, percentage denuded endothelium, and percentage of fenestrated endothelium. Light microscopy was used to measure mean glomerular volume, total filtration surface per glomerulus, cortical interstitial fractional volume per glomerulus, and percentage of sclerotic glomeruli (Squarer et al. 1998).

Statistical analyses. Change in urine adenine was expressed as a fraction of the urine adenine concentration measured closest to the kidney biopsy and the urine adenine concentration measured a year prior to the biopsy. The same approach was used for change in the urine albumin:creatinine ratio. Variables with a skewed distribution were log transformed prior to analyses. Cross-sectional relationships between change in urine adenine and each morphometric variable were assessed by Pearson correlations and after adjustment for age and sex by linear regression and partial Pearson correlations.

Chronic Renal Insufficiency Cohort (CRIC). The parent CRIC Study recruited (from 2003 on) a racially diverse group aged 21 to 74 years, ˜50% diabetic, with a broad range of kidney function. Informed consent was obtained from participants; protocols were approved by IRBs and Scientific and Data Coordinating Center (approval #807882). The current study analyzed the urine metabolome at study entry (baseline) of 995 randomly selected CRIC participants with diabetes across CKD stages 3a, 3b, and 4, eGFR 45-60, 30-45, and 20-30 ml/min/1.73 m 2, respectively.

Disease outcomes. Two outcomes were evaluated: annual rate of eGFR change (eGFR slope) and time-to-ESKD. EGFR-slope was estimated via mixed models using serial eGFR measures as described previously. Time-to-ESKD was the time from entry to CRIC Study to incident kidney failure with need for renal replacement therapy or kidney transplantation; drop-out or death before kidney failure were considered censoring events.

The results from a multi-omic unbiased analysis using spatial metabolomics, untargeted urine metabolomics, single cell transcriptomics and single nuclear transcriptomics from kidney biopsies and urine samples identified that the polyamine pathway is implicated in tubular function. A specific metabolite, adenine, is prognostic for kidney disease progression in patients with diabetes. Further, urine adenine is a robust prognostic biomarker especially in patients with diabetes and normoalbuminuria, which is a large population of patients with diabetes.

Urine samples obtained 6 months to 1 year prior to the kidney biopsy was analyzed for urine albumin/creatinine ratios and urine adenine/creatinine (by the ZipChip-QE method). 15 subjects were identified who had normal GFR and before they were treated with medications that affected the renin-angiotensin system.

As noted, the GFR was above the range of kidney disease (<90 ml/min). The urine albumin/creatinine ratio (ACR) ranged from normal levels (<30 mg albumin/gram creatinine) to the high range (>300 mg albumin/gram creatinine). The urine ACR and urine adenine/creatinine ratio was measured on two separate occasions prior to the kidney biopsy and the change in urine ACR or the change in urine adenine/creatinine ratio was statistically correlated with the degree of pathology in the kidney biopsy. Surprisingly, the change in urine albumin/creatinine ratio did not show a significant correlation with a common marker of established kidney disease (glomerulosclerosis, r=0.22, p=0.17), however the change in urine adenine/creatinine ratio did show a significant correlation with the degree of glomerulosclerosis (r=0.57, p=0.03).

Another marker of kidney disease is the expansion of the interstitial volume (non-glomerular region). There was a poor and insignificant correlation with urine albumin/creatinine ratio and cortical interstitial fractional volume (r=0.057, p=0.83) whereas there was a trend to a correlation between urine adenine/creatinine and the cortical interstitial fractional volume (r=0.38, p=0.16). The cumulative data indicates that the change in urine adenine/creatinine ratio identifies which patients have underlying kidney pathology indicating kidney disease, with normal levels of GFR.

The urine adenine/creatinine ratio can identify which patients with diabetes and normoalbuminuria and normal GFR will have a rapid decline in GFR. The PIMA Native American cohort of 60 patients who had urine adenine levels measured prior to the kidney biopsy were divided into tertiles (t1 3-67; t2 72-411; t3 429-2954 nmole adenine/nmole creatinine) based on their urine adenine levels prior to the kidney biopsy. The patients were divided into those with normoalbuminuria (n=32), microalbuminuria (n=16) or macroalbuminuria (n=12). Their GFR was measured was measured annually throughout the trial by the urinary clearance of iothalamate. Statistical analysis was performed with the outcome being >=40% decline in GFR (using our standard imputation technique) with a median follow-up of 7.6 years (IQR 3.8-11.8). 47 patients of the 60 participants met the criteria for GFR decline and a decline of >40% in GFR is an acceptable FDA criteria for clinical decline in kidney function (PMID 25446026). The participants (n=62, mean age 45 f 10 years) had a baseline mean f standard deviation glomerular filtration rate of 137 f 50 ml/min and median (interquartile range) urine albumin:creatinine ratio of 34 (14-85) mg/g near the time of the biopsy (PMID 30830355).

The urine adenine levels based on the upper tertile vs the lowest tertile was highly predictive of measured GFR decline over a 7.6 year follow period in patients with normoalbuminuria (HR=4.3, 95% CI 1.14-16.21, p=0.03, n=32). Of note, the GFR was measured with inulin-based measurements and is thus a measure of true GFR. There was also a significant relationship in the comparison of Tertile 2 vs Tertile 1 in the microalbuminuria group.

This data indicates that diabetic patients with normoalbuminuria and G3 category of CKD will have a 4.4-fold higher risk of kidney failure if they are in the upper tertile of urine adenine as compared to the lowest tertile. The diabetic patients with normoalbuminuria and G3 category of CKD will have a 3-fold higher risk of kidney failure if they are in the second tertile of urine adenine as compared to the lowest tertile.

Urine adenine could identify which diabetic patients will require treatment in the G1A1 stage of kidney disease. Once a diagnosis of patients at high risk of kidney disease progression for decline of GFR>40% while in the G1 stage, the clinician can readily select a treatment regimen that is compatible with the diagnosis such as selecting certain anti-hypertensive medications (renin-angiotensin inhibitors), mineralocorticoid receptor blockers, certain medications that affect blood glucose levels (such as sodium-glucose transporter inhibitors (SGLT2i) or glucagon-like peptide agonists (GLP1 agonists). All of these medications may reduce the progression of kidney disease but would be warranted primarily in patients at high risk of developing progressive decline in kidney function. Additional dietary interventions such as altering sodium, protein, fat and carbohydrates may be of benefit for the high risk patients. Changing lifestyle such as stopping smoking or increasing exercise may also be beneficial for this high risk population that could not otherwise be identified.

Urine adenine could identify which diabetic patients will require treatment in the G3A1 stage of kidney disease. Once a diagnosis of patients at high risk of kidney failure while in the G3A1 stage, the clinician can readily select a treatment regimen that is compatible with the diagnosis such as selecting certain anti-hypertensive medications (renin-angiotensin inhibitors), mineralocorticoid receptor blockers, certain medications that affect blood glucose levels (such as sodium-glucose transporter inhibitors (SGLT2i) or glucagon-like peptide agonists (GLP1 agonists). All of these medications may reduce the progression of kidney disease but would be warranted primarily in patients at high risk of developing progressive decline in kidney function. Additional dietary interventions such as altering sodium, protein, fat and carbohydrates may be of benefit for the high risk patients. Changing lifestyle such as stopping smoking or increasing exercise may also be beneficial for this high risk population that could not otherwise be identified.

### Example 3

### Using Zipchip Assay to Measure Adenine in a Biofluid

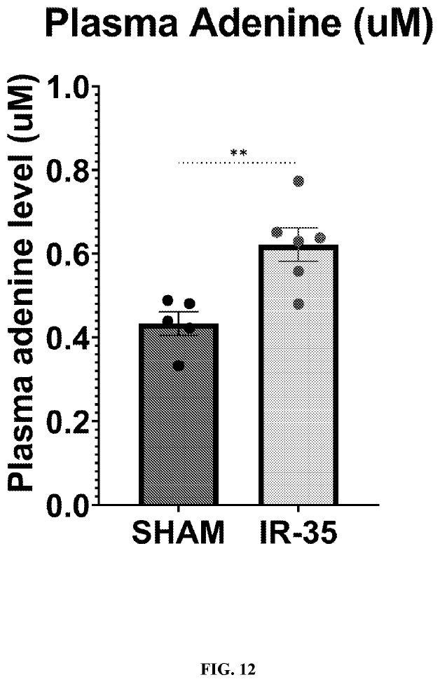

In FIG. 12, plasma adenine was measured by Zipchip-QExactive method in mice 24 h post sham surgery or 24 h post ischemia-reperfusion injury. There is a significant increase in plasma adenine in the IRI mice indicating they would be a good model for mimicking human kidney disease. With the ischemia-reperfusion model, the plasma adenine was found to be elevated in the mice after 24 h post ischemia-perfusion (n=5) as compared to the sham-operated mice (n=5).

Urine adenine identifies animal model could be used for modeling chronic kidney injury. Animal models may be useful to mimic human diseases. By demonstrating that adenine is involved in human kidney disease, the measurement of plasma or urine adenine could be used to identify relevant models of progressive kidney disease. With the Zucker diabetic fatty rat model, urine adenine/creatinine ratio was measured in n=9 ZDF rats and in the lean control rats n=9. There was a four-fold increase in urine adenine/creatinine ratio in the ZDF rats (2603 nM/mM) as compared to control rats (502 nM/mM).

### EXAMPLE 4

### Protocol of Using Zipchip™ to Measure Adenine Levels

A. All steps below should be performed in ice bath.

B. Adenine stock solution at 1000 μM and creatinine stock solution at 3000 μM are prepared in MS grade water and stored in −80° C. freezer.

C. Preparation of calibration standards.

In the day of sample preparation, mix 20 μL of adenine stock and 20 μL of creatinine stock to obtain a mixture containing 500 μM adenine and 1500 μM creatinine.

Dilute as below to obtain desired concentrations (only show adenine concentrations).

| | Beginning | Std | H2O | |
|---|---|---|---|---|
| Final | std conc | Volume | Volume | Volumn |
| conc (μM) | (μM) | (μL) | (μL) | left (μL) |
| 0.00 (H2O) | | | | |
| 0.01 | 0.1 | 5.0 | 45.0 | 50.0 |
| 0.1 | 1 | 5.0 | 45.0 | 45.0 |
| 1 | 2.5 | 10.0 | 15.0 | 20.0 |
| 2.5 | 5 | 15.0 | 15.0 | 20.0 |
| 5 | 10 | 15.0 | 15.0 | 15.0 |
| 10 | 20 | 15.0 | 15.0 | 15.0 |
| 20 | 50 | 16.0 | 24.0 | 25.0 |
| 50 | 100 | 16.0 | 16.0 | 16.0 |
| 100 | 200 | 15.0 | 15.0 | 14.0 |
| 200 | 300 | 20.0 | 10.0 | 15.0 |
| 300 | 400 | 24.0 | 8.0 | 12.0 |
| 400 | 500 | 28.0 | 7.0 | 11.0 |
| 500 | 1000 | 20.0 | 20.0 | 12.0 |

Dilute as below to obtain desired concentrations (only show adenine concentrations).

D. Preparation of extraction solution with internal standards.

80 mL MeOH+10 mL 1M NH4Ac+10 mL H2O+25 μL of 25 mM creatinine d3+5 μL of 15 mM adenine-13C. Mix well, aliquot 5 mL each and store at −80° C. until use.

E. In the day of analysis, thaw urine samples and pooled human urine as QC sample in ice bath.

F. Prepare 96 well plate (Thermo, AB-0800) with proper labeling and sample/QC location template.

G. Vortex each sample and transfer 10 μL each to the desired well. Transfer 10 μL QC sample into wells A1, C3, E5, G7, and D9.

H. Transfer 10 μL of calibration standards into proper wells (C11 to H12).

I. Using a multiple channel pipette, transfer 90 μL of extraction solution (D above) into each well of samples, QC sample, calibration standards, and blank.

J. Cover the plate and keep in −20° C. freezer for at least 30 min.

K. Centrifuge the plate at 9000 rpm, 4° C., for 8-10 mins.

L. Using multiple channel pipette, transfer top 50 μL from each well to a fresh plate. Cover the plate with “Zone free” (Sigma, Z721646-50EA) plate cover.

M. Instrument: Thermo Scientific Q Exactive HFX and 908 Devices ZipChip interface CE/MS.

N. Prepare BGE following the manufacturer's instruction (908 Devices). A full bottle BGE is needed for a full 96 well plate samples.

O. Check mass spectrometer for operational conditions. Perform mass calibration.

P. Prime autosampler and HSX chip, check chip spray.

Q. Set up sequences for both MS and ZipChip systems to acquire data, starting with blank, then lowest concentration of calibration standards.

| | |
|---|---|
| ZipChip Method: | Field Strength: 1000 V/cm |
| | Load Volume: 5 nL |
| | Chip Type: HS |
| | BGE: Metabolite |
| | Pressure Assis: no |
| | Run Time: 4 minutes |
| MS Settings: | m/z range: 70-500 |
| | Resolution: 15,000 |
| | AGC target: 1E6 |
| | Max Ion Injection Time: 20 ms |
| | Inlet Capillary Temperature 200° C. |
| | S Lens RF: 40 |
| | Run Time: 4 minutes |

R. Perform “Rinse Sample Well” 8 times after the highest concentration calibration and before first sample injection to eliminate carryover.

S. Process data with Thermo Scientific's software Xcalibur-Quan Browser to obtain adenine and creatinine concentrations for each urine sample and QC sample.

### Example 5

### High Urine Adenine Identifies Non-Macroalbuminuric Patients Who are at High Risk of ESKD Outcomes

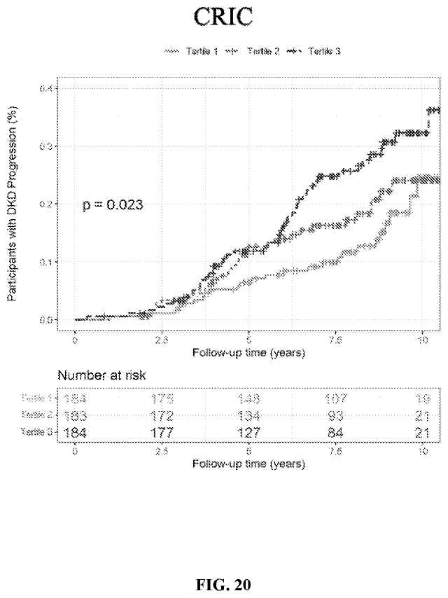

Patients with diabetes are often categorized as normoalbuminuric (0-30 mg albumin/gm creatinine), microalbuminuric (30-300 mg/g) or macroalbuminuric (>300 mg/g). As patients with normo- or microalbuminuria could revert to either category, this group of patients could be considered as non-macroalbuminuric (<300 mg/g). In new analysis we have also found that when patients are categorized as non-macroalbuminuric (i.e. <300 mg albumin/gram creatinine) there is an increased risk of ESKD defined as >50% decline in eGFR, need for renal replacement therapy or persistent eGFR<15 ml/min/1.73m2 (FIG. 20). This non-macroalbuminuric categorization encompasses >90% of patients with diabetes in the United States.

### Example 6

### High Urine Adenine Identifies Diabetic Patients at Risk of ESKD and all Cause Mortality

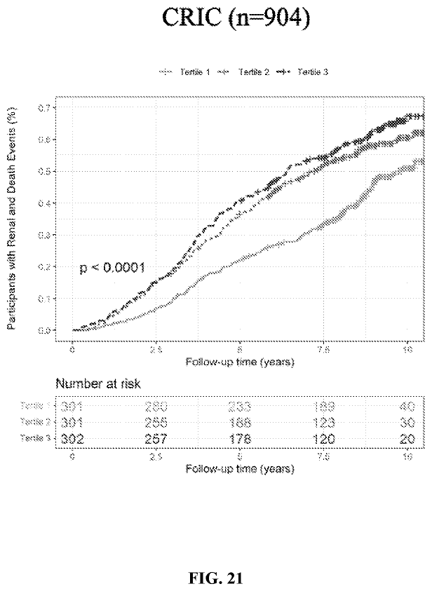

Another finding was that high urine adenine levels predict ESKD and all-cause mortality independent of albuminuria status in a US study with n=904 participants (FIG. 21). Patients who were in the upper two tertiles have a HR of 1.6-1.8 vs lowest tertile for ESKD and all-cause mortality. This data indicates that measurement of urine adenine/creatinine levels will provide clinical value for all patients with diabetes regardless of albuminuria status (FIG. 21).

### Example 7

### High Urine Adenine is a Non-Invasive Marker of Hyperfiltration in Diabetic Patients

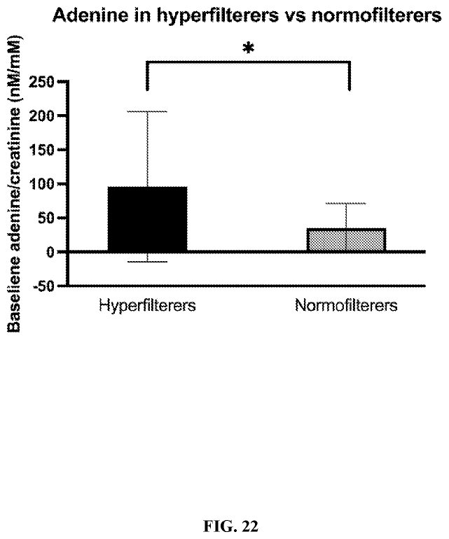

In patients with diabetes, the earliest risk factor for kidney disease is a physiologic finding called hyperfiltration, however this measurement is difficult to make without an invasive protocol which requiring intravenous infusions. The function of the normal kidney is measured by the glomerular filtration rate (GFR) and the GFR is accurately measured by an intravenous infusion of certain molecules (iothalamate, inulin, etc.) that is completely filtered by the glomerulus and minimally reabsorbed or secreted at the tubular level. The normal GFR varies between 90-120 ml/min/1.73m2. With the initial diagnosis of either type 1 or type diabetes, there is a complex change in blood flow regulation in the kidney leading to an elevated GFR (>135 ml/min/1.73m2 termed hyperfiltration), in the patients who are at risk of progressive kidney disease. This is thought to be one of the earliest markers of diabetic kidney disease and occurs before an increase in albuminuria. However, measuring GFR is very difficult in patients and therefore a non-invasive marker would be very useful. Another new finding is that patients with type 1 diabetes and hyperfiltration have high urine adenine values as compared to patients with type 1 diabetes who have normal GFR (FIG. 22).

### Example 8

a Treatment that is Protective for Kidney and Heart Disease Reduces Urine Adenine Levels and the Urine Adenine/Creatinine Ratio could be Used as A Pharmacodynamic or Surrogate Biomarker

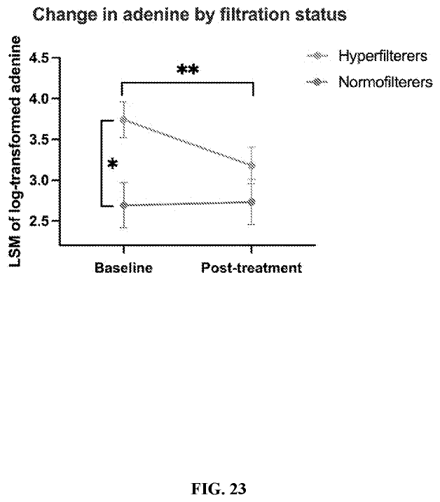

Urine adenine/creatinine ratio may be used to identify patients who respond to SGLT2 inhibitors as Empagliflozin reduces urine adenine/creatinine levels in high risk patients with hyperfiltration after 8 weeks of treatment. (FIG. 23).

### Example 9

### Treatments to Reduce Adenine Production Protects Against Kidney Disease

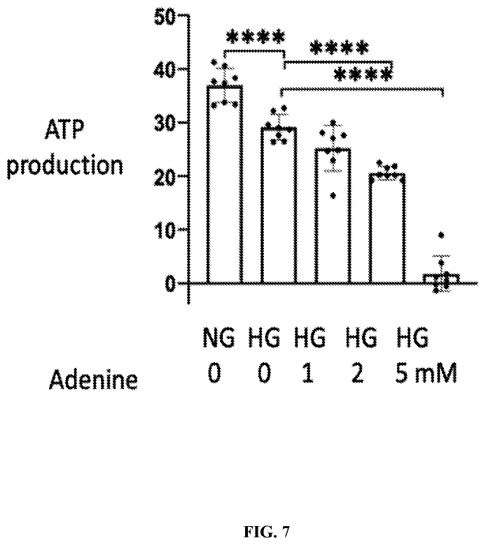

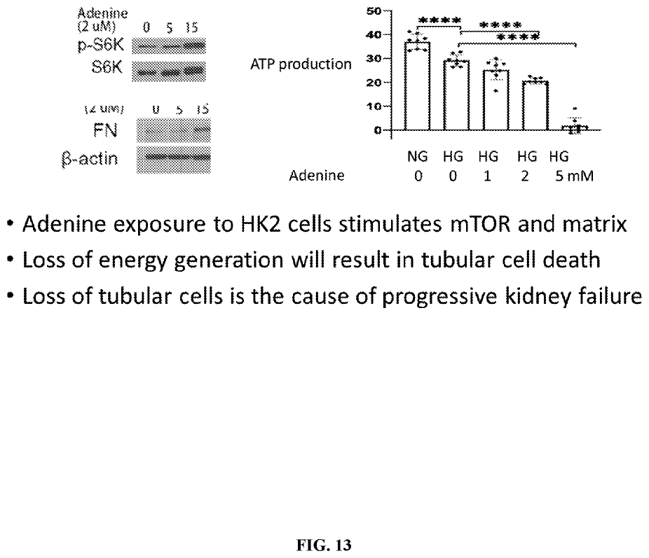

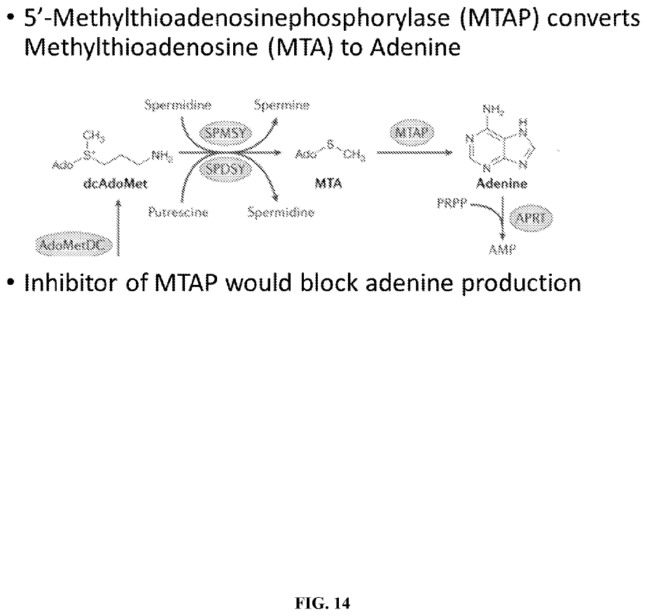

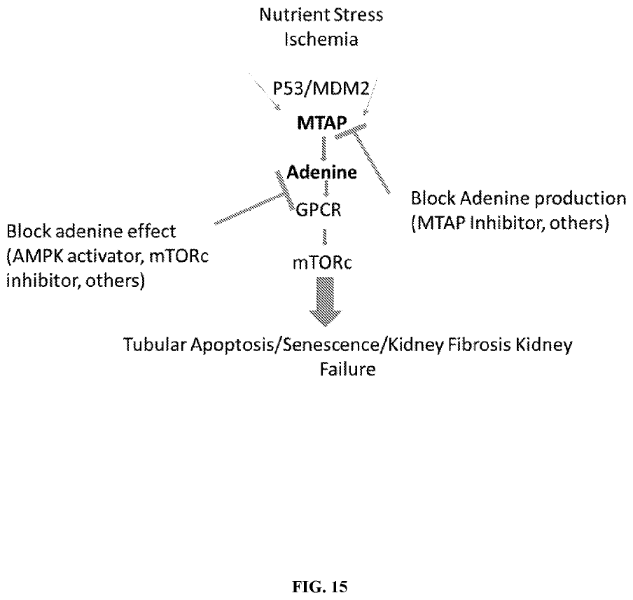

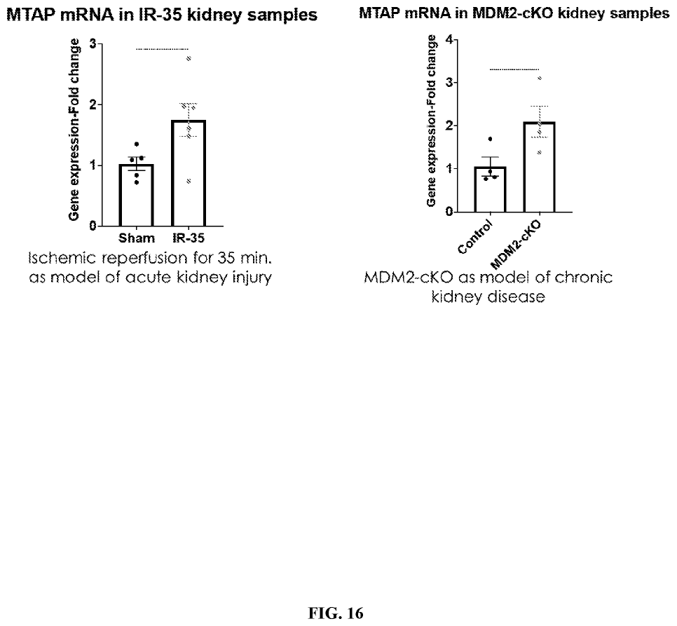

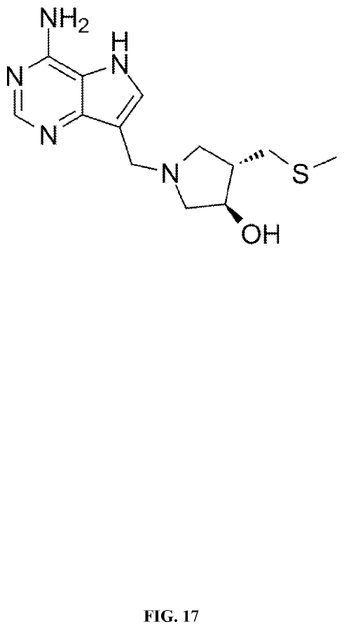

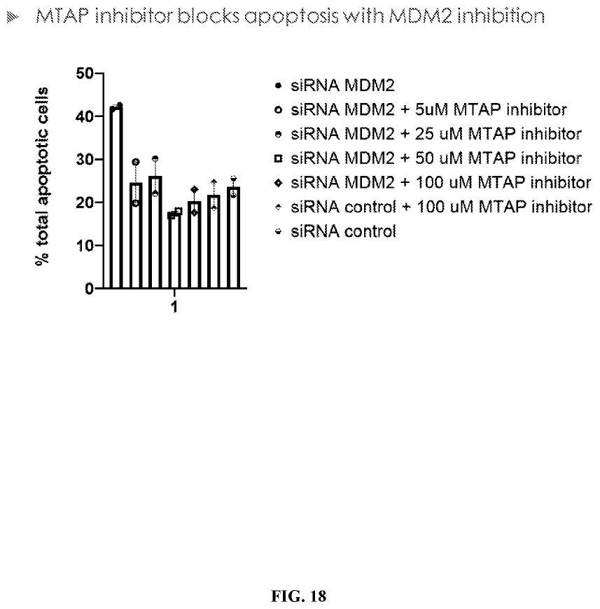

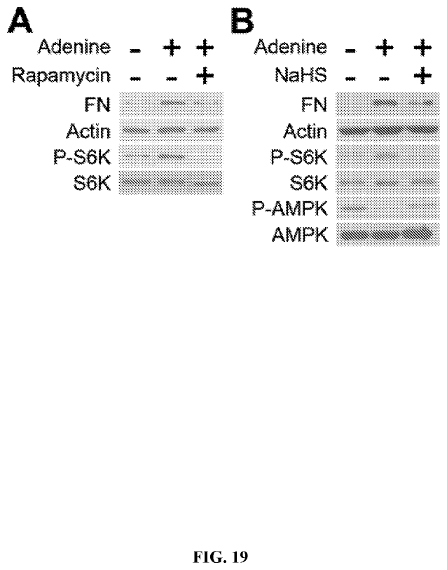

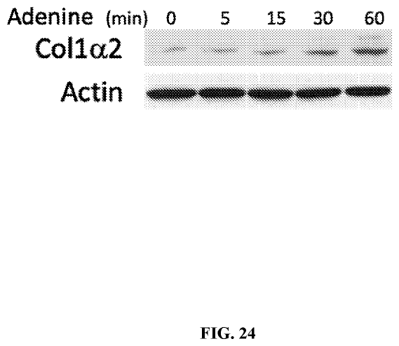

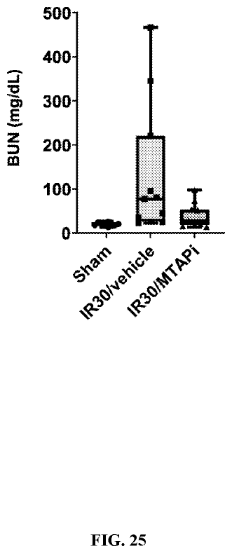

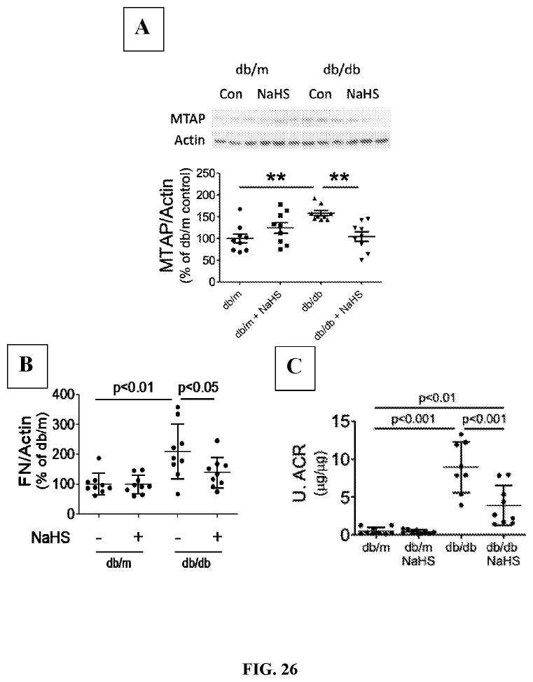

Adenine was found to be toxic to kidney cells (FIG. 7 and FIG. 13) and stimulates extracellular matrix molecules (type I collagen and fibronectin production in kidney tubular cells (FIG. 13 and FIG. 24). The production of extracellular matrix molecules is known to contribute to fibrosis and provides rationale as to why adenine is correlated with glomerulosclerosis in patients (FIG. 8). A major source of endogenous adenine production is via the MTAP pathway (FIG. 14). Therefore, conditions in which MTAP is increased would indicate that these conditions will benefit from inhibition of MTAP (FIG. 15) and be protective to the kidney. MTAP gene expression was increased in the acute kidney injury (AKI) model of ischemia-reperfusion (FIG. 16 left panel) and in the MDM2 tubular deficient model of chronic kidney disease (FIG. 16 right panel). A small molecule specific inhibitor of MTAP (DADMe-ImmA) was administered for the first time in a model of AKI and found to be protective of kidney function as measured by BUN levels (FIG. 25). The small molecule MTAP inhibitor, DADMe-ImmA was also protective to kidney cells under conditions of cell stress (due to reduction of MDM2) (FIG. 18). Another novel approach to inhibit MTAP is via the compound Sodium Hydrogen Sulfide (NaHS). Administering NaHS to kidney cells protects against adenine-induced matrix production and disease causing pathways (mTOR) while improving healing pathways (AMPK) (FIG. 19 right panel). Treatment with NaHS to db/db mice (a model of diabetic kidney disease) led to reduction of MTAP (FIG. 24) in the diabetic kidney and improvement in ACR and matrix accumulation in the kidney.

### Example 10

Inhibition of the Insulin Receptor with Novel siRNA Blocks Adenine Induced Stimulation of MTOR and Matrix Production

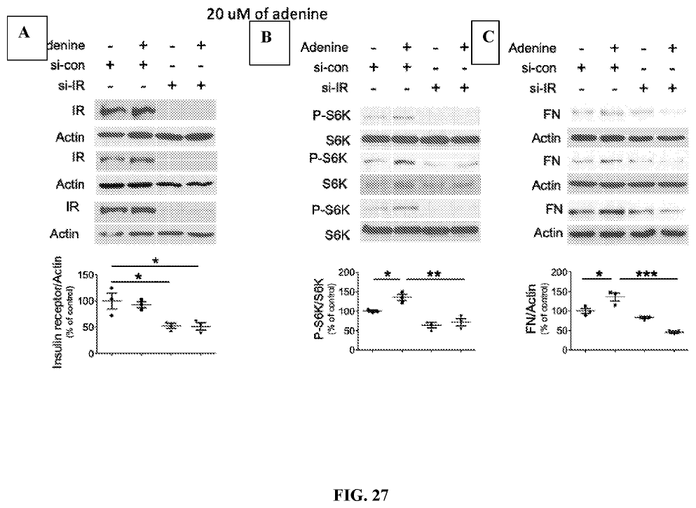

The receptor by which adenine causes intracellular signaling is via the insulin receptor as inhibition of the insulin receptor with a novel siRNA completely blocks adenine signaling and matrix production (FIG. 27).

### Example 11

### Treatments to Inhibit Downstream Pathways of Adenine is Protective of Kidney Cells

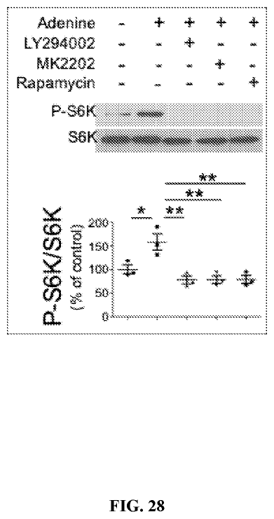

The downstream pathways stimulated by adenine are PI3Kinase, AKT and mTOR (FIG. 28). Inhibition with small molecule inhibitor of PI3K (LY294002), AKT (MK2202) or mTOR (Rapamycin) all are effective to reduce mTOR activation in kidney tubular cells. Once mTOR is inhibited the downstream effect of adenine to stimulate matrix accumulation was reduced (FIG. 19).

### Example 12

### Inhibition of Adenine Production is Protective Against Diabetic Kidney Disease

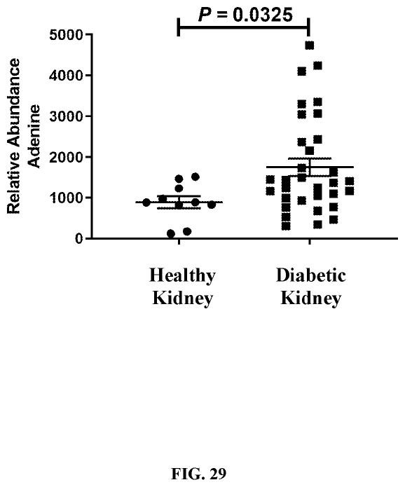

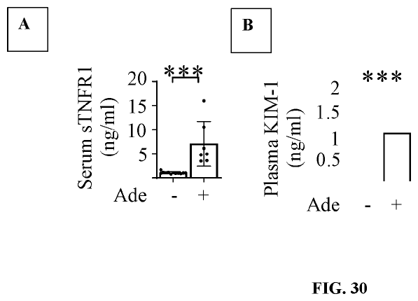

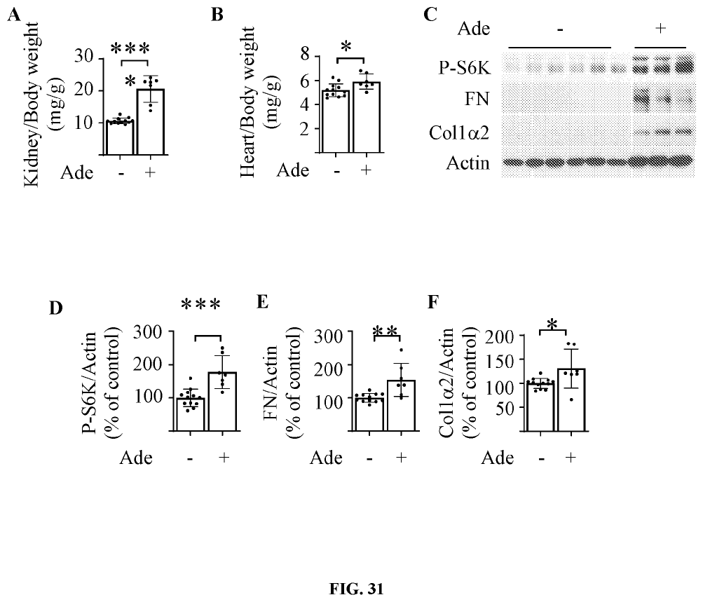

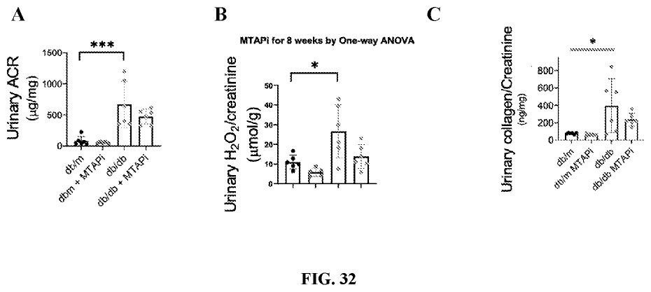

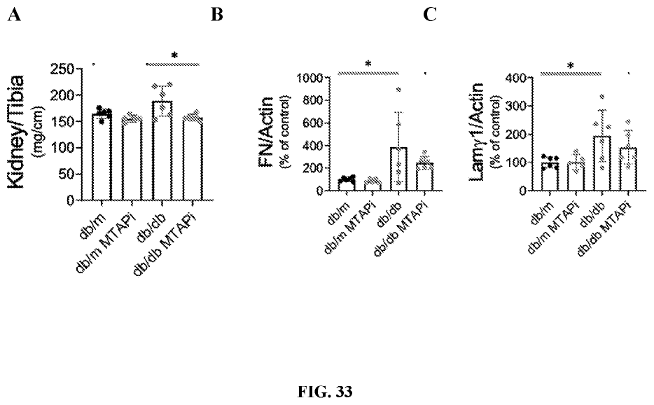

Patients with type 1 diabetes (n=8) or type 2 diabetes (n=8) have an increase in tissue levels of adenine in their kidney biopsies as compared to healthy controls (n=5)(FIG. 29). Adenine administration is causative of the major manifestations of diabetic kidney disease in a mouse model, including serum soluble tumor necrosis factor receptor-1 (sTNFR1) and plasma kidney injury marker-1 (KIM1) (FIG. 30). Adenine also stimulates kidney hypertrophy and heart hypertrophy (FIG. 31) as well as kidney phosphor-S6-kinase (marker of mTOR activity), kidney fibronectin and kidney collagen type 1 alpha-2 chain (FIG. 31). An inhibitor of MTAP (DADMe-ImmA 10 mg/kg/d in drinking water for 8 weeks) reduces urine ACR, urine hydrogen peroxide (urine H2O2) and urine collagen in a mouse model of diabetic kidney disease (db/db mouse) (FIG. 32). The MTAP inhibitor also reduced kidney hypertrophy, kidney fibronectin and kidney laminin yI (FIG. 33).

### Example 13

siRNA for Mouse and Rat Insulin Receptor can Reduce Mouse Insulin Receptor in Mouse Tubular Cells

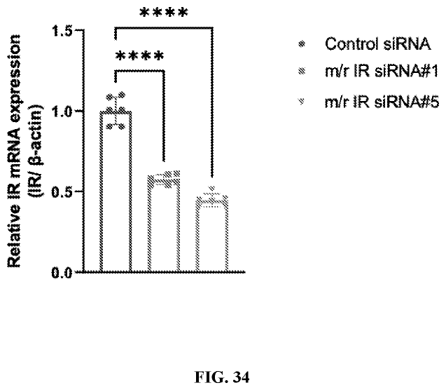

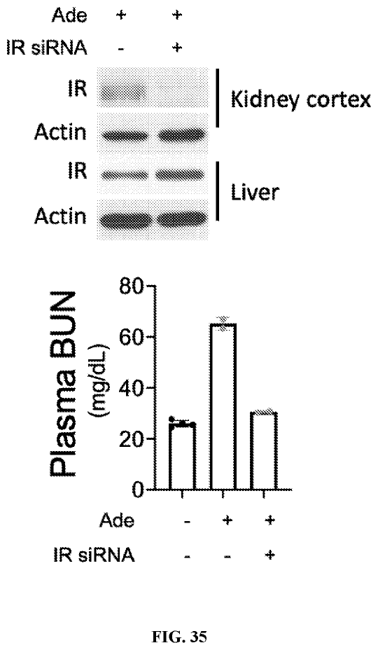

FIG. 34 illustrates the administration of the siRNA for the insulin receptor to mice treated with adenine reduced insulin receptor in the kidney but not in liver and prevented the increase in blood urea nitrogen levels (FIG. 35). In FIG. 34, siRNAs to insulin receptor (IR) based on sequence in mouse and rat reduces IR in mouse kidney cells. Mouse cortical tubular (MCT) cells were reverse transfected with siRNAs targeting mouse and rat insulin receptor mRNA (m/r IRsiRNA #1 and m/r IR siRNA #5) using RNAiMax (6 ul in optiMEM) and plated on 6 well plates. After 72 h of incubation, total RNA was isolated and subjected to cDNA synthesis followed by qRTPCR (n=6/group, ****p<0.0001). There was a significant reduction of mouse insulin receptor at mRNA level with both IR siRNA #1 and IRsiRNA #5.

In FIG. 35, in vivo administration of IRsiRNA #1 reduced IR specifically in mouse kidney and prevented adenine-induced rise in Blood Urea Nitrogen (BUN) levels in mice. Mice were administered vehicle or kidney targeting IRsiRNA #1 prior to being challenged with adenine (5 mM) in the drinking water for 4 days. At 4 days, mouse kidney showed reduction of IR in mouse kidney cortex, as compared to control sample, but not in liver as demonstrated by immunostaining with antibody to IR and b-actin (representative immunoblot, upper panel). The mice given vehicle and adenine (n=2) had an elevation in BUN levels of 3-fold vs non-adenine group (lower panel). The mice treated with siRNA for IR had no increase in BUN levels despite exposure to adenine (n=2) (lower panel). Table 10A shows example sequences of the siRNAs that were and could be used to target mouse or rat IR mRNA; however it is contemplated that other siRNAs targeting IR mRNA, including those commercially available can also be used.

TABLE 10A
Example siRNA sequences targeting mouse/rat IR mRNA.

| si RNA | Sequence ID No. and sequence | Strand |
|---|---|---|
| M/R IR | SEQ ID NO. 1 | + |
| siRNA#1 | 5′ rGrGrCrUrCrUrGrUrCrCrGrCrArUrCrGrArGrArArGrArATA 3′ | |
| | SEQ ID NO. 2 | − |
| | 5′ rUrArUrUrCrUrUrCrUrCrGrArUrGrCrGrGrArCrArGrArGrCrCrCrC 3′ | |
| M/R IR | SEQ ID NO. 3 | + |
| siRNA#2 | 5′ rGrUrCrArUrUrGrUrCrArGrArArArGrUrUrUrGrCrCrCrAAC 3′ | |
| | SEQ ID NO. 4 | − |
| | 5′ rGrUrUrGrGrGrCrArArArCrUrUrUrCrUrGrArCrArArUrGrArCrUrG 3′ | |
| M/R IR | SEQ ID NO. 5 | + |
| siRNA#3 | 5′ rUrUrCrArGrGrArArGrCrUrArCrArUrCrUrGrArUrUrCrGAG 3′ | |
| | SEQ ID NO. 6 | − |
| | 5′ rCrUrCrGrArArUrCrArGrArUrGrUrArGrCrUrUrCrCrUrGrArArGrA 3′ | |
| M/R IR | SEQ ID NO. 7 | + |
| siRNA#4 | 5′ rCrArArArGrGrArArCrCrUrArArUrGrGrUrCrUrGrArUrUGT 3′ | |
| | SEQ ID NO. 8 | − |
| | 5′ rArCrArArUrCrArGrArCrCrArUrUrArGrGrUrUrCrCrUrUrUrGrGrC 3′ | |
| M/R IR | SEQ ID NO. 9 | + |
| siRNA#5 | 5′ rArArArUrCrArGrUrUrCrCrUrCrArArArUrUrGrArCrCrAAT 3′ | |
| | SEQ ID NO. 10 | − |
| | 5′ rArUrUrGrGrUrCrArArUrUrUrGrArGrGrArArCrUrGrArUrUrUrUrG 3′ | |

TABLE 10B
Targeting sequences of the siRNAs in Table 10A.

| si RNA | Targeting sequence | SEQ ID No. |
|---|---|---|
| M/R IR | GGCTCTGTCCGCATCGAGAAGAATA | SEQ ID No. 23 |
| siRNA#1 | | |
| M/R IR | GTCATTGTCAGAAAGTTTGCCCAAC | SEQ ID No. 24 |
| siRNA#2 | | |
| M/R IR | TTCAGGAAGCTACATCTGATTCGAG | SEQ ID No. 25 |
| siRNA#3 | | |
| M/R IR | CAAAGGAACCTAATGGTCTGATTGT | SEQ ID No. 26 |
| siRNA#4 | | |
| M/R IR | AAATCAGTTCCTCAAATTGACCAAT | SEQ ID No. 27 |
| siRNA#5 | | |

siRNA Targeting Human/Pig Insulin Receptor

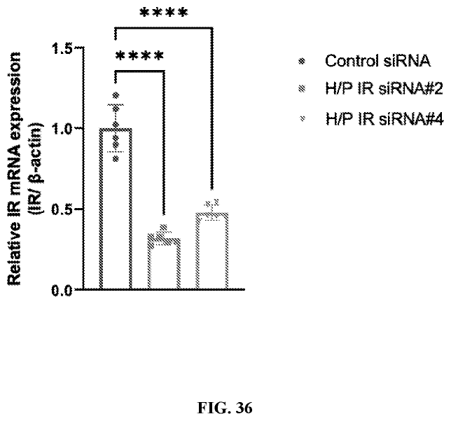

In this non-limiting example, siRNA targeting human/pig insulin receptor mRNA can reduce human insulin receptor in human tubular cells. (FIG. 36). In FIG. 36, siRNA to human and pig insulin receptor is effective to reduce mRNA for insulin receptor in human kidney cells. Human kidney (HK2) proximal tubular cells were transfected with siRNA (4 ul from 10 um stock) using RNAiMax (6 ul in optiMEM) and plated on 6 well plates. After 48 h, the media was replaced with fresh media. After 96 h of the transfection, total RNA was isolated and subjected to cDNA synthesis followed by qRTPCR. There was a significant reduction of human insulin receptor in HK2 cells after transfection with H/P siRNA #2 and H/P IR siRNA #4 as compared to control siRNA (n=6/group, ***p<0.0001). Table 11 shows example sequences of the siRNAs that were and could be used to target human or pig IR mRNA; however it is contemplated that other siRNAs targeting IR mRNA, including those commercially available can also be used.

TABLE 11A
Example siRNA sequences targeting human and pig IR mRNA.

| Example | | Strand |
|---|---|---|
| si RNA | Sequence ID No. and sequence | |
| H/P | SEQ ID NO. 11 | + |
| siRNA#1A | 5′ rArGrUrUrUrCrCrCrCrArArArCrUrCrArUrCrArUrGrArUCA 3′ | |
| | SEQ ID NO. 12 | − |
| | 5′ rUrGrArUrCrArUrGrArUrGrArGrUrUrUrGrGrGrGrArArArCrUrGrA 3′ | |
| H/P | SEQ ID NO. 13 | + |
| siRNA#1B | 5′ rUrCrArUrGrArUrCrArCrUrGrArUrUrArCrUrUrGrCrUrGCT 3′ | |
| | SEQ ID NO. 14 | − |
| | 5′ rArGrCrArGrCrArArGrUrArArUrCrArGrUrGrArUrCrArUrGrArUrG 3′ | |
| H/P | SEQ ID NO. 15 | + |
| siRNA#1C | 5′ rGrArUrCrArCrUrGrArUrUrArCrUrUrGrCrUrGrCrUrCrUTC 3′ | |
| | SEQ ID NO. 16 | − |
| | 5′ rGrArArGrArGrCrArGrCrArArGrUrArArUrCrArGrUrGrArUrCrArU 3′ | |
| H/P | SEQ ID NO. 17 | + |
| siRNA#2 | 5′ rCrArGrUrArCrGrUrCrArUrUrCrArCrArArCrArArCrArAGT 3′ | |
| | SEQ ID NO. 18 | − |
| | 5′ rArCrUrUrGrUrUrGrUrUrGrUrGrArArUrGrArCrGrUrArCrUrGrGrU 3′ | |
| H/P | SEQ ID NO. 19 | + |
| siRNA#3 | 5′ rGrUrCrUrUrUrCrUrCrUrUrCrArGrUrGrUrUrGrUrGrArUTG 3′ | |
| | SEQ ID NO. 20 | − |
| | 5′ rCrArArUrCrArCrArArCrArCrUrGrArArGrArGrArArArGrArCrArA 3′ | |
| H/P | SEQ ID NO. 21 | + |
| siRNA#4 | 5′ rGrGrArArUrGrArCrCrArGrArGrArCrArUrCrUrArUrGrAAA 3′ | |
| | SEQ ID NO. 22 | − |
| | 5′ rUrUrUrCrArUrArGrArUrGrUrCrUrCrUrGrGrUrCrArUrUrCrCrArA 3′ | |

TABLE 11B
Targeting sequences of the siRNAs in Table 11A.

| | | SEQ |
|---|---|---|
| si RNA | Targeting sequence | ID No. |
| H/P siRNA#1A- | AGTTTCCCCAAACTCATCATGATCA | SEQ ID |
| 1C | | No. 28 |
| H/P siRNA#2 | CAGTACGTCATTCACAACAACAAGT | SEQ ID |
| | | No. 29 |
| H/P siRNA#3 | GTCTTTCTCTTCAGTGTTGTGATTG | SEQ ID |
| | | No. 30 |
| H/P siRNA#4 | GGAATGACCAGAGACATCTATGAAA | SEQ ID |
| | | No. 31 |

1. A method of identifying a subject at risk of a progressive fibrosis or cellular senescence comprising:
(a) measuring a level of a nucleic acid, nucleoside, or amino acid in a biological sample from the subject; and
(b) assessing the risk of the kidney disease based on the nucleic acid, nucleoside, or amino acid level as compared to a standard.

2. The method of claim 1, wherein the nucleic acid, nucleoside or amino acid is a purine, polyamine, adenine, cytosine, guanine, thymine, uracil, asparagine, aspartic acid, betaine, homocysteine, isoleucine, L-alpha-aminobutyric acid, lysine, methionine, nicotinic acid, ornithine, phenylalanine, pipecolate, threonine, tryptophan, or valine, or any combination thereof.

3. The method of claim 1, wherein the kidney disease is at least one of chronic kidney disease, diabetic kidney disease, hypertension-related kidney disease, glomerulonephritis-associated kidney disease, end-stage renal disease (kidney failure), glomerulosclerosis, tubulointerstitial fibrosis, kidney arterial sclerosis, kidney arteriolar sclerosis, kidney neoplasia, or kidney tubular atrophy.

4. The method of claim 1, wherein the risk of kidney disease comprises risk of disease progression.

5. The method of claim 1, wherein the risk of kidney disease comprises risk of developing the kidney disease in the future.

6. The method of claim 1, further comprises processing a biological sample from the subject to separate or enrich the nucleic acid, nucleoside, or amino acid.

7. The method of claim 6, wherein processing the biological sample comprises performing capillary electrophoresis, liquid chromatography (HPLC), capillary electrophoresis, or liquid chromatograph, or any combination thereof.

8. The method of claim 1, wherein measuring the nucleic acid, nucleoside, or amino acid level is by diode array detection, mass spectrometry, electromagnetic radiation absorption, or ZipChip™.

9. The method of claim 8, wherein the diode Array detection is performed using the wavelengths between 200-400 nm.

10. The method of claim 1, wherein the nucleic acid measured is adenine, and wherein a adenine level of greater than a threshold value is indicative of decline in glomerular filtration rate (GFR), CKD progression, and/or kidney failure, wherein the threshold value is about 2.92 nmol adenine/mmol creatinine, about 4.08 nmol adenine/mmol creatinine, or about 5.23 nmol adenine/mmol creatinine.

11. The method of claim 1, wherein the nucleic acid measured is adenine and wherein an adenine level greater than about 2.92 nmol adenine/mmol creatinine, about 4.08 nmol adenine/mmol creatinine, or about 5.23 nmol adenine/mmol creatinine is indicative of ESKD.

12. The method of claim 1, wherein the subject is diabetic.

13. The method of claim 1, wherein the subject has normal albumin levels.

14. The method of claim 1, wherein the subject is a mammal.

15. (canceled)

16. The method of claim 1, further comprising administering a treatment to the subject, wherein the treatment comprises surgery, chemotherapy, radiation therapy, dietary restrictions, treatment of high blood pressure, treatment of diabetes, weight management, smoking cessation, treatment of high cholesterol and/or other lipid levels, kidney transplant, dialysis, administration of erythropoietin and/or calcitriol, diuretics, vitamin D, or phosphate binder, pharmaceutical composition inhibiting production or function of adenine, or any combination thereof.

17. The method of claim 1, wherein the biological sample is urine or blood.

18. A method of treating or preventing a kidney disease in a subject having or at risk of developing a kidney disease comprising:
(a) determining the level of adenine in a biological sample from the subject; and
(b) administering a treatment for the kidney disease if the adenine level is above about 2.92 nmol adenine/mmol creatinine, about 4.08 nmol adenine/mmol creatinine, or about 5.23 nmol adenine/mmol creatinine.

19. (canceled)

20. (canceled)

21. (canceled)

22. (canceled)

23. (canceled)

24. (canceled)

25. An assay for determining the level of adenine in a biological sample from a subject comprising:
(a) separating analytes in a sample forming sample fractions; and
(b) quantifying adenine in the appropriate fractions.

26. The assay of claim 25, wherein the biological sample is urine or blood.

27.-84. (canceled)


SEQUENCE LISTING (31 sequences)

SEQ ID NO: 1 - RNA, 25 nt, synthetic construct
  source 1..25: mol_type = other RNA
ggctctgtccgcatcgagaagaata

SEQ ID NO: 2 - RNA, 27 nt, synthetic construct
  source 1..27: mol_type = other RNA
tattcttctcgatgcggacagagcccc

SEQ ID NO: 3 - RNA, 25 nt, synthetic construct
  source 1..25: mol_type = other RNA
gtcattgtcagaaagtttgcccaac

SEQ ID NO: 4 - RNA, 27 nt, synthetic construct
  source 1..27: mol_type = other RNA
gttgggcaaactttctgacaatgactg

SEQ ID NO: 5 - RNA, 25 nt, synthetic construct
  source 1..25: mol_type = other RNA
ttcaggaagctacatctgattcgag

SEQ ID NO: 6 - RNA, 27 nt, synthetic construct
  source 1..27: mol_type = other RNA
ctcgaatcagatgtagcttcctgaaga

SEQ ID NO: 7 - RNA, 25 nt, synthetic construct
  source 1..25: mol_type = other RNA
caaaggaacctaatggtctgattgt

SEQ ID NO: 8 - RNA, 27 nt, synthetic construct
  source 1..27: mol_type = other RNA
acaatcagaccattaggttcctttggc

SEQ ID NO: 9 - RNA, 25 nt, synthetic construct
  source 1..25: mol_type = other RNA
aaatcagttcctcaaattgaccaat

SEQ ID NO: 10 - RNA, 27 nt, synthetic construct
  source 1..27: mol_type = other RNA
attggtcaatttgaggaactgattttg

SEQ ID NO: 11 - RNA, 25 nt, synthetic construct
  source 1..25: mol_type = other RNA
agtttccccaaactcatcatgatca

SEQ ID NO: 12 - RNA, 27 nt, synthetic construct
  source 1..27: mol_type = other RNA
tgatcatgatgagtttggggaaactga

SEQ ID NO: 13 - RNA, 25 nt, synthetic construct
  source 1..25: mol_type = other RNA
tcatgatcactgattacttgctgct

SEQ ID NO: 14 - RNA, 27 nt, synthetic construct
  source 1..27: mol_type = other RNA
agcagcaagtaatcagtgatcatgatg

SEQ ID NO: 15 - RNA, 25 nt, synthetic construct
  source 1..25: mol_type = other RNA
gatcactgattacttgctgctcttc

SEQ ID NO: 16 - RNA, 27 nt, synthetic construct
  source 1..27: mol_type = other RNA
gaagagcagcaagtaatcagtgatcat

SEQ ID NO: 17 - RNA, 25 nt, synthetic construct
  source 1..25: mol_type = other RNA
cagtacgtcattcacaacaacaagt

SEQ ID NO: 18 - RNA, 27 nt, synthetic construct
  source 1..27: mol_type = other RNA
acttgttgttgtgaatgacgtactggt

SEQ ID NO: 19 - RNA, 25 nt, synthetic construct
  source 1..25: mol_type = other RNA
gtctttctcttcagtgttgtgattg

SEQ ID NO: 20 - RNA, 27 nt, synthetic construct
  source 1..27: mol_type = other RNA
caatcacaacactgaagagaaagacaa

SEQ ID NO: 21 - RNA, 25 nt, synthetic construct
  source 1..25: mol_type = other RNA
ggaatgaccagagacatctatgaaa

SEQ ID NO: 22 - RNA, 27 nt, synthetic construct
  source 1..27: mol_type = other RNA
tttcatagatgtctctggtcattccaa

SEQ ID NO: 23 - DNA, 25 bp, synthetic construct
  source 1..25: mol_type = other DNA
ggctctgtccgcatcgagaagaata

SEQ ID NO: 24 - DNA, 25 bp, synthetic construct
  source 1..25: mol_type = other DNA
gtcattgtcagaaagtttgcccaac

SEQ ID NO: 25 - DNA, 25 bp, synthetic construct
  source 1..25: mol_type = other DNA
ttcaggaagctacatctgattcgag

SEQ ID NO: 26 - DNA, 25 bp, synthetic construct
  source 1..25: mol_type = other DNA
caaaggaacctaatggtctgattgt

SEQ ID NO: 27 - DNA, 25 bp, synthetic construct
  source 1..25: mol_type = other DNA
aaatcagttcctcaaattgaccaat

SEQ ID NO: 28 - DNA, 25 bp, synthetic construct
  source 1..25: mol_type = other DNA
agtttccccaaactcatcatgatca

SEQ ID NO: 29 - DNA, 25 bp, synthetic construct
  source 1..25: mol_type = other DNA
cagtacgtcattcacaacaacaagt

SEQ ID NO: 30 - DNA, 25 bp, synthetic construct
  source 1..25: mol_type = other DNA
gtctttctcttcagtgttgtgattg

SEQ ID NO: 31 - DNA, 25 bp, synthetic construct
  source 1..25: mol_type = other DNA
ggaatgaccagagacatctatgaaa